(19) United States
(12) Patent Application Publication
(10) Pub. No.: US 2010/0331357 A1
(43) Pub. Date: Dec. 30, 2010

(54) PHARMACEUTICAL COMPOSITION

(75) Inventors: Tomomichi Futo, Osaka (JP); Shinichiro Nakai, Osaka (JP); Rinaldo Laurentius De Jong, San Diego, CA (US); Bumsup Lee, San Diego, CA (US); Ronald Joseph Christopher, Carlsbad, CA (US)

Correspondence Address: WENDEROTH, LIND & PONACK, L.L.P., 1030 15th Street, N.W.,, Suite 400 East, Washington, DC, 20005-1503, US

(73) Assignee: TAKEDA PHARMACEUTICAL COMPANY LIMITED, Osaka-shi, Osaka (JP)

(21) Appl. No.: 12/918,505

(22) PCT Filed: Feb. 27, 2009
(86) PCT No.: PCT/JP2009/054237

§ 371 Date: Aug. 31, 2010

Related U.S. Application Data
(60) Provisional application No. 61/032,165, filed on Feb. 28, 2008.

(51) Int. Cl. A61K 31/437; A61P 35/00; A61P 29/00; A61P 1/00; A61P 17/06; A61P 25/00; A61P 25/16; A61P 25/28

(52) U.S. Cl. 514/292

(57) ABSTRACT

The present invention provides a pharmaceutical composition, wherein solubility and stability of a water-insoluble or slightly water-soluble compound represented by formula (I): wherein each symbol is as defined in the specification, are improved, by combination of the above-mentioned compound and a cyclodextrin derivative and a method for improving solubility, stability and the like of the above-mentioned compound.

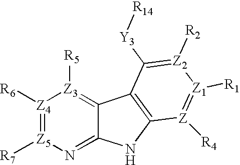

### TECHNICAL FIELD

The present invention relates to a pharmaceutical composition wherein solubility or stability of a water-insoluble or slightly water-soluble compound is improved, and a method for improving solubility or stability of the compound.

### BACKGROUND OF THE INVENTION

WO2007/044779 and US2007/0117816 describe that a compound represented by the formula:

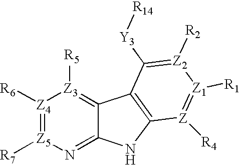

wherein
Z, Z₁, Z₂, Z₃, Z₄ and Z₅ are each independently selected from the group consisting of C and N;
R₁ is —Y₁—R₁₂, or R₁ is absent when Z₁ is N;
R₂ is —Y₂—R₁₃, or R₂ is absent when Z₂ is N, or R₁ and R₂ are taken together to form a ring;
Y₁, Y₂ and Y₃ are each independently absent or a linker providing 1 or 2 atom separation between R₁₂, R₁₃ or R₁₄ and the ring to which Y₁, Y₂ or Y₃ is attached, wherein the atoms of the linker providing the separation are selected from the group consisting of carbon, oxygen, nitrogen, and sulfur;
R₄ is selected from the group consisting of hydrogen, halo, nitro, cyano, thio, oxy, hydroxy, carbonyloxy, alkoxy, carbonyl, amino, (C₁₋₅)alkylamino, (C₁₋₅)alkyl, halo(C₁₋₅)alkyl, carbonyl(C₁₋₃)alkyl, sulfonyl(C₁₋₃)alkyl, amino(C₁₋₅)alkyl, aryl(C₁₋₅)alkyl, heteroaryl(C₁₋₅)alkyl, (C₃₋₆)cycloalkyl and hetero(C₃₋₆)cycloalkyl, each substituted or unsubstituted, with the proviso that R₄ is absent when the atom to which it is bound is N;
R₅ and R₆ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloaklyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloalkyl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, with the proviso that each of R₅ and R₆ is absent when the atom to which it is bound is N;
R₇ is selected from the group consisting of hydrogen, halo, hydroxy, alkoxy, amino and (C₁₋₅)alkyl, each substituted or unsubstituted, with the proviso that R₇ is absent when the atom to which it is bound is N;
R₁₂ and R₁₃ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₂ and R₁₃ are taken together to form a ring; and
R₁₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted,
   with the provisos that (a) —Y₃—R₁₄ is not H when Z, Z₁, Z₂, Z₃ and Z₅ are all C; R₅ is a substituted amino group; and R₂ is methoxy or R₇ is methyl or amino; and (b) R₁₄ is not 3-chlorophenyl when R₁, R₅, R₆ and R₇ are each H; Z and Z₂ are each N; R₂ and R₄ are absent; Z₁, Z₃, Z₄ and Z₅ are all C; and Y₃ is NH,
   a salt thereof and a prodrug thereof, have a Kinase-inhibiting effect and are useful as a prophylactic and therapeutic agent against the diseases including cancer, inflammation, inflammatory bowel disease, psoriasis, transplant rejection, amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss, contraceptive medication, mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, androgenetic alopecia, dementia related diseases, Alzheimer's Disease, conditions associated with kinases, arthritis and the like.

### DISCLOSURE OF THE INVENTION

The present invention aims at providing a pharmaceutical composition, wherein solubility, stability and the like of the above-mentioned compound are improved, and a method for improving solubility, stability and the like of the above-mentioned compound.

In view of the above-mentioned problems, the present inventors have conducted intensive studies and unexpectedly succeeded in obtaining a pharmaceutical composition, wherein solubility, stability and the like of said compound are markedly improved, by combination of the above-mentioned compound and a cyclodextrin derivative. Based on this finding, the present inventors have further investigated and completed the present invention.

Accordingly, the present invention provides

[1] a pharmaceutical composition comprising;
i) a compound represented by formula (I):

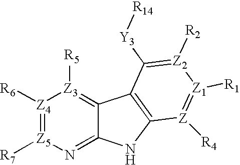

wherein
Z, Z₁, Z₂, Z₃, Z₄ and Z₅ are each independently selected from the group consisting of C and N;
R₁ is —Y₁—R₁₂, or R₁ is absent when Z₁ is N;
R₂ is —Y₂—R₁₃, or R₂ is absent when Z₂ is N, or R₁ and R₂ are taken together to form a substituted or unsubstituted ring;
Y₁, Y₂ and Y₃ are each independently absent or a linker providing 1 or 2 atom separation between R₁₂, R₁₃ or R₁₄ and the ring to which Y₁, Y₂ or Y₃ is attached, wherein the atoms of the linker providing the separation are selected from the group consisting of carbon, oxygen, nitrogen and sulfur;
R₄ is selected from the group consisting of hydrogen, halo, nitro, cyano, thio, oxy, hydroxy, carbonyloxy, alkoxy, carbonyl, amino, (C₁₋₅)alkylamino, (C₁₋₅)alkyl, halo(C₁₋₅)alkyl, carbonyl(C₁₋₃)alkyl, sulfonyl(C₁₋₃)alkyl, amino(C₁₋₅)alkyl, aryl(C₁₋₅)alkyl, heteroaryl(C₁₋₅)alkyl, (C₃₋₆)cycloalkyl and hetero(C₃₋₆)cycloalkyl, each substituted or unsubstituted, with the proviso that R₄ is absent when the atom to which it is bound is N;
R₅ and R₆ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, with the proviso that each of R₅ and R₆ is absent when the atom to which it is bound is N;
R₇ is selected from the group consisting of hydrogen, halo, hydroxy, alkoxy, amino and (C₁₋₅)alkyl, each substituted or unsubstituted, with the proviso that R₇ is absent when the atom to which it is bound is N;
R₁₂ and R₁₃ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₂ and R₁₃ are taken together to form a substituted or unsubstituted ring; and
R₁₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃) alkyl, sulfonyl(C₁₋₃)alkyl, sulfinyl(C₁₋₃)alkyl, amino(C₁₋₁₀) alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted,
   with the provisos that (a) —Y₃—R₁₄ is not H when Z, Z₁, Z₂, Z₃ and Z₅ are all C; R₅ is a substituted amino group; and R₂ is methoxy or R₇ is methyl or amino; and (b) R₁₄ is not 3-chlorophenyl when R₁, R₅, R₆ and R₇ are each H; Z and Z₂ are each N; R₂ and R₄ are absent; Z₁, Z₃, Z₄ and Z₅ are all C; and Y₃ is NH,
   a salt thereof or a prodrug thereof,
   ii) at least one cyclodextrin derivative and
   iii) water,
   [2] the composition of the above-mentioned [1], wherein the compound represented by the formula (I) described in the above-mentioned [1] is 5-(3-(ethylsulfonyl)phenyl)-3,8-dimethyl-N-(1-methylpiperidin-4-yl)-9H-pyrido[2,3-b]indole-7-carboxamide,
   [3] the composition of the above-mentioned [1], which is used as an injectable composition,
   [4] the composition of the above-mentioned [1], which is a non-emulsified composition,
   [5] the composition of the above-mentioned [1], further comprising at least one pharmaceutically acceptable substance selected from a solubilizer, a suspending agent, an isotonic agent, a buffering agent, a local anesthetics and a pH adjusting agent,
   [6] the composition of the above-mentioned [1], wherein pH of the composition is 2 to 5,
   [7] the composition of the above-mentioned [1], which comprises about 0.01 to about 10 mol of the cyclodextrin derivative per 1 mol of the compound of the formula (I) described in the above-mentioned [1],
   [8] the composition of the above-mentioned [1], wherein the cyclodextrin derivative is at least one selected from a hydroxyalkyl cyclodextrin, a glucosyl cyclodextrin, a maltosyl cyclodextrin and a sulfoalkyl ether cyclodextrin,
   [9] the composition of the above-mentioned [1], wherein the cyclodextrin derivative is at least one selected from a hydroxyalkyl cyclodextrin, a glucosyl cyclodextrin, a maltosyl cyclodextrin and a sulfoalkyl ether cyclodextrin, and a salt thereof,
   [10] the composition of the above-mentioned [1], wherein the cyclodextrin derivative is sulfobutyl ether-β-cyclodextrin sodium salt,
   [11] the composition of the above-mentioned [1], wherein the content of the cyclodextrin derivative in the pharmaceutical composition is about 0.01 to about 90 w/v %,
   [12] the composition of the above-mentioned [1], which is an agent for preventing or treating of cancer, inflammation, inflammatory bowel disease, psoriasis, transplant rejection, amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss, contraceptive medication, mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment, cognitive impairment, androgenetic alopecia, dementia related diseases, Alzheimer's Disease or conditions associated with kinases,
   [13] a pharmaceutical composition comprising;
   i) 5-(3-(ethylsulfonyl)phenyl)-3,8-dimethyl-N-(1-methylpiperidin-4-yl)-9H-pyrido[2,3-b]indole-7-carboxamide or a salt thereof,
   ii) sulfobutyl ether-β-cyclodextrin or a salt thereof, and
   iii) water,
   [14] a pharmaceutical composition comprising;
   i) 5-(3-(ethylsulfonyl)phenyl)-3,8-dimethyl-N-(1-methylpiperidin-4-yl)-9H-pyrido[2,3-b]indole-7-carboxamide or a salt thereof,
   ii) sulfobutyl ether-β-cyclodextrin or a salt thereof,
   iii) water and
   iv) citric acid,
   which is an injectable composition, having a pH of about 2 to about 4,
   [15] the composition of the above-mentioned [14], which is prepared by a method comprising;
   i) dissolving a) 5-(3-(ethylsulfonyl)phenyl)-3,8-dimethyl-N-(1-methylpiperidin-4-yl)-9H-pyrido[2,3-b]indole-7-carboxamide or a salt thereof, b) sulfobutyl ether-β-cyclodextrin or a salt thereof and c) citric acid n water to obtain a solution, and ii) adjusting pH of the solution to about 2 to about 4,
   [16] a pharmaceutical composition comprising;
   i) 5 to 40 mg/mL of 5-(3-(ethylsulfonyl)phenyl)-3,8-dimethyl-N-(1-methylpiperidin-4-yl)-9H-pyrido[2,3-b]indole-7-carboxamide or a salt thereof,
   ii) 2 to 30 w/v % of sulfobutyl ether-β-cyclodextrin or a salt thereof,
   iii) water and
   iv) 10 to 100 mmol/L of citric acid,
   which is an injectable composition, having a pH of about 2 to about 4,
   [17] the composition of any one of the above-mentioned [13]-[16], wherein the sulfobutyl ether-β-cyclodextrin or a salt thereof is sulfobutyl ether-β-cyclodextrin sodium salt,
   [18] a pharmaceutical composition comprising;
   i) a compound represented by formula (I):

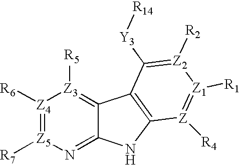

wherein
Z, Z₁, Z₂, Z₃, Z₄ and Z₅ are each independently selected from the group consisting of C and N;
R₁ is —Y₁—R₁₂, or R₁ is absent when Z₁ is N;
R₂ is —Y₂—R₁₃, or R₂ is absent when Z₂ is N, or R₁ and R₂ are taken together to form a substituted or unsubstituted ring;
Y₁, Y₂ and Y₃ are each independently absent or a linker providing 1 or 2 atom separation between R₁₂, R₁₃ or R₁₄ and the ring to which Y₁, Y₂ or Y₃ is attached, wherein the atoms of the linker providing the separation are selected from the group consisting of carbon, oxygen, nitrogen and sulfur;
R₄ is selected from the group consisting of hydrogen, halo, nitro, cyano, thio, oxy, hydroxy, carbonyloxy, alkoxy, carbonyl, amino, (C₁₋₅)alkylamino, (C₁₋₅)alkyl, halo(C₁₋₅)alkyl, carbonyl(C₁₋₃)alkyl, sulfonyl(C₁₋₃)alkyl, amino(C₁₋₅)alkyl, aryl(C₁₋₅)alkyl, heteroaryl(C₁₋₅)alkyl, (C₃₋₆)cycloalkyl and hetero(C₃₋₆)cycloalkyl, each substituted or unsubstituted, with the proviso that R₄ is absent when the atom to which it is bound is N;
R₅ and R₆ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, with the proviso that each of R₅ and R₆ is absent when the atom to which it is bound is N;
R₇ is selected from the group consisting of hydrogen, halo, hydroxy, alkoxy, amino and (C₁₋₅)alkyl, each substituted or unsubstituted, with the proviso that R₇ is absent when the atom to which it is bound is N;
R₁₂ and R₁₃ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₂ and R₁₃ are taken together to form a substituted or unsubstituted ring; and
R₁₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀) heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, with the provisos that (a) —Y₃—R₁₄ is not H when Z, Z₁, Z₂, Z₃ and Z₅ are all C; R₅ is a substituted amino group; and R₂ is methoxy or R₇ is methyl or amino; and (b) R₁₄ is not 3-chlorophenyl when R₁, R₅, R₆ and R₇ are each H; Z and Z₂ are each N; R₂ and R₄ are absent; Z₁, Z₃, Z₄ and Z₅ are all C; and Y₃ is NH,
   a salt thereof or a prodrug thereof, and
   ii) sulfobutyl ether-β-cyclodextrin sodium salt,
   [19] a pharmaceutical composition comprising;
   i) 5-(3-(ethylsulfonyl)phenyl)-3,8-dimethyl-N-(1-methylpiperidin-4-yl)-9H-pyrido[2,3-b]indole-7-carboxamide or a salt thereof, and
   ii) sulfobutyl ether-β-cyclodextrin or a salt thereof,
   [20] a pharmaceutical composition comprising;
   i) 5-(3-(ethylsulfonyl)phenyl)-3,8-dimethyl-N-(1-methylpiperidin-4-yl)-9H-pyrido[2,3-b]indole-7-carboxamide or a salt thereof, and
   ii) sulfobutyl ether-β-cyclodextrin sodium salt,
   [21] a method for preventing or treating cancer, inflammation, inflammatory bowel disease, psoriasis, transplant rejection, amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss, contraceptive medication, mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment, cognitive impairment, androgenetic alopecia, dementia related diseases, Alzheimer's Disease or conditions associated with kinases, which comprises administrating an effective amount of the composition of the above-mentioned [1] to a mammalian species in need thereof,
   [22] a method of improving solubility in water of the compound of the formula (I) described in the above-mentioned [1], a salt thereof or a prodrug thereof, which comprises combining the compound of the formula (I), a salt thereof or a prodrug thereof with at least one cyclodextrin derivative,
   [23] a method of improving stability in water of the compound of the formula (I) described in the above-mentioned [1], a salt thereof or a prodrug thereof, which comprises combining the compound of the formula (I), a salt thereof or a prodrug thereof with at least one cyclodextrin derivative.

### DEFINITIONS

Unless otherwise stated, the following terms used in the specification and claims shall have the following meanings for the purposes of this Application.

“Alicyclic” means a moiety comprising a non-aromatic ring structure. Alicyclic moieties may be saturated or partially unsaturated with one, two or more double or triple bonds. Alicyclic moieties may also optionally comprise heteroatoms such as nitrogen, oxygen and sulfur. The nitrogen atoms can be optionally quaternized or oxidized and the sulfur atoms can be optionally oxidized. Examples of alicyclic moieties include, but are not limited to moieties with C₃₋₈ rings such as cyclopropyl, cyclohexane, cyclopentane, cyclopentene, cyclopentadiene, cyclohexane, cyclohexene, cyclohexadiene, cycloheptane, cycloheptene, cycloheptadiene, cyclooctane, cyclooctene, and cyclooctadiene.

“Aliphatic” means a moiety characterized by a straight or branched chain arrangement of constituent carbon atoms and may be saturated or partially unsaturated with one, two or more double or triple bonds.

“Alkoxy” means an oxygen moiety having a further alkyl substituent. The alkoxy groups of the present invention can be optionally substituted.

“Alkyl” represented by itself means a straight or branched, saturated or unsaturated, aliphatic radical having a chain of carbon atoms, optionally with oxygen (See “oxaalkyl”), oxoalkyl (See “oxoalkyl”), sulfur (See “thioalkyl”), or nitrogen atoms (See “azaalkyl”) between the carbon atoms. C_{X} alkyl and C_{X-Y} alkyl are typically used where X and Y indicate the number of carbon atoms in the chain. For example, C₁₋₆ alkyl includes alkyls that have a chain of between 1 and 6 carbons (e.g., methyl, ethyl, propyl, isopropyl, butyl, sec-butyl, isobutyl, tert-butyl, vinyl, allyl, 1-propenyl, isopropenyl, 1-butenyl, 2-butenyl, 3-butenyl, 2-methylallyl, ethynyl, 1-propynyl, 2-propynyl, and the like). Alkyl represented along with another radical (e.g., as in arylalkyl, heteroarylalkyl) means a straight or branched, saturated or unsaturated aliphatic divalent radical having the number of atoms indicated or when no atoms are indicated means a bond (e.g., (C₆₋₁₀)aryl(C₁₋₃)alkyl includes benzyl, phenethyl, 1-phenylethyl, 3-phenylpropyl, 2-thienylmethyl, 2-pyridinylmethyl and the like).

“Alkenyl” means a straight or branched, carbon chain that contains at least one carbon-carbon double bond. Examples of alkenyl include vinyl, allyl, isopropenyl, pentenyl, hexenyl, heptenyl, 1-propenyl, 2-butenyl, 2-methyl-2-butenyl, and the like.

“Alkynyl” means a straight or branched, carbon chain that contains at least one carbon-carbon triple bond. Examples of alkynyl include ethynyl, propargyl, 3-methyl-1-pentynyl, 2-heptynyl and the like.

“Alkylene”, unless indicated otherwise, means a straight or branched, saturated or unsaturated, aliphatic, divalent radical. C_{X} alkylene and C_{X-Y} alkylene are typically used where X and Y indicate the number of carbon atoms in the chain. For example, C₁₋₆ alkylene includes methylene (—CH₂—), ethylene (—CH₂CH₂—), trimethylene (—CH₂CH₂CH₂—), tetramethylene (—CH₂CH₂CH₂CH₂—), 2-butenylene (—CH₂CH═CHCH₂—), 2-methyltetramethylene (—CH₂CH(CH₃)CH₂CH₂—), pentamethylene (—CH₂CH₂CH₂CH₂CH₂—) and the like.

“Alkenylene” means a straight or branched, divalent carbon chain having one or more carbon-carbon double bonds. Examples of alkenylene include ethene-1,2-diyl, propene-1,3-diyl, methylene-1,1-diyl, and the like.

“Alkynylene” means a straight or branched, divalent carbon chain having one or more carbon-carbon triple bonds. Examples of alkynylene include ethyne-1,2-diyl, propyne-1,3-diyl, and the like.

“Alkylidene” means a straight or branched saturated or unsaturated, aliphatic radical connected to the parent molecule by a double bond. C_{X} alkylidene and C_{X-Y} alkylidene are typically used where X and Y indicate the number of carbon atoms in the chain. For example, C₁₋₆ alkylidene includes methylidene (═CH₂), ethylidene (═CHCH₃), isopropylidene (═C(CH₃)₂), propylidene (═CHCH₂CH₃), allylidene (═CH—CH═CH₂), and the like.

“Amino” means a nitrogen moiety having two further substituents where, for example, a hydrogen or carbon atom is attached to the nitrogen. For example, representative amino groups include —NH₂, —NHCH₃, —N(CH₃)₂, —NH(C₁₋₁₀)_{alkyl}, —N((C₁₋₁₀)alkyl₂, —NHaryl, —NHheteroaryl, —N(aryl)₂, —N(heteroaryl)₂, and the like. Optionally, the two substituents together with the nitrogen may also form a ring. Unless indicated otherwise, the compounds of the invention containing amino moieties may include protected derivatives thereof. Suitable protecting groups for amino moieties include acetyl, tert-butoxycarbonyl, benzyloxycarbonyl, and the like.

“Azaalkyl” means an alkyl, as defined above, except where one or more of the carbon atoms forming the alkyl chain are replaced with substituted or unsubstituted nitrogen atoms (—N—). For example, a (C₁₋₁₀)azaalkyl refers to a chain comprising between 1 and 10 carbons and one or more nitrogen atoms.

“Animal” includes humans, non-human mammals (e.g., dogs, cats, rabbits, cattle, horses, sheep, goats, swines, deer, and the like) and non-mammals (e.g., birds, and the like).

“Aromatic” means a moiety wherein the constituent atoms make up an unsaturated ring system, all atoms in the ring system are sp² hybridized and the total number of pi electrons is equal to 4n+2. An aromatic ring may be such that the ring atoms are only carbon atoms or may include carbon and non-carbon atoms (see “heteroaryl”).

“Aryl” means a monocyclic or polycyclic ring assembly wherein each ring is aromatic or when fused with one or more rings forms an aromatic ring assembly. If one or more ring atoms is not carbon (e.g., N, S), the aryl is a heteroaryl. C_{X} aryl and C_{X-Y} aryl are typically used where X and Y indicate the number of carbon atoms in the ring.

“Bicycloalkyl” means a saturated or partially unsaturated fused bicyclic or bridged polycyclic ring assembly.

“Bicycloaryl” means a bicyclic ring assembly wherein the rings are linked by a single bond or fused and at least one of the rings comprising the assembly is aromatic. C_{X} bicycloaryl and C_{X-Y} bicycloaryl are typically used where X and Y indicate the number of carbon atoms in the bicyclic ring assembly and directly attached to the ring.

“Bridging ring” as used herein refers to a ring that is bonded to another ring to form a compound having a bicyclic structure where two ring atoms that are common to both rings are not directly bound to each other. Non-exclusive examples of common compounds having a bridging ring include borneol, norbornane, 7-oxabicyclo[2.2.1]heptane, and the like. One or both rings of the bicyclic system may also comprise heteroatoms.

“Carbamoyl” means the radical —C(O)NRₐR_{b} where Rₐ and R_{b} are each independently two further substituents and comprise a hydrogen or carbon atom is attached to the nitrogen.

“Carbocycle” means a ring consisting of carbon atoms.

“Carbocyclic ketone derivative” means a carbocyclic derivative wherein the ring contains a —CO— moiety.

“Carbonyl” means the radical —CO—. It is noted that the carbonyl radical may be further substituted with a variety of substituents to form different carbonyl groups including acids, acid halides, aldehydes, amides, esters, and ketones.

“Carboxy” means the radical —CO₂—. It is noted that compounds of the invention containing carboxy moieties may include protected derivatives thereof, i.e., where the oxygen is substituted with a protecting group. Suitable protecting groups for carboxy moieties include benzyl, tert-butyl, and the like.

“Cyano” means the radical —CN.

“Cycloalkyl” means a non-aromatic, saturated or partially unsaturated, monocyclic, fused bicyclic or bridged polycyclic ring assembly. C_{X} cycloalkyl and C_{X-Y} cycloalkyl are typically used where X and Y indicate the number of carbon atoms in the ring assembly. For example, C₃₋₁₀ cycloalkyl includes cyclopropyl, cyclobutyl, cyclopentyl, cyclohexyl, cyclohexenyl, 2,5-cyclohexadienyl, bicyclo[2.2.2]octyl, adamantan-1-yl, decahydronaphthyl, oxocyclohexyl, dioxocyclohexyl, thiocyclohexyl, 2-oxobicyclo[2.2.1]hept-1-yl, and the like.

“Cycloalkylene” means a divalent saturated or partially unsaturated, monocyclic or polycyclic ring assembly. C_{X} cycloalkylene and C_{X-Y} cycloalkylene are typically used where X and Y indicate the number of carbon atoms in the ring assembly.

“Disease” specifically includes any unhealthy condition of an animal or part thereof and includes an unhealthy condition that may be caused by, or incident to, medical or veterinary therapy applied to that animal, i.e., the “side effects” of such therapy.

“Fused ring” as used herein refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems may be saturated or partially unsaturated, carbocyclics, heterocyclics, aromatics, heteroaromatics, or the like.

“Halo” means fluoro, chloro, bromo or iodo.

“Halo-substituted alkyl”, as an isolated group or part of a larger group, means “alkyl” substituted by one or more “halo” atoms, as such terms are defined above. Halo-substituted alkyl includes haloalkyl, dihaloalkyl, trihaloalkyl, perhaloalkyl and the like (e.g., halo-substituted (C₁₋₃)alkyl includes chloromethyl, dichloromethyl, difluoromethyl, trifluoromethyl, 2,2,2-trifluoroethyl, perfluoroethyl, 2,2,2-trifluoro-1,1-dichloroethyl, and the like).

“Heteroatom” refers to an atom that is not a carbon atom. Particular examples of heteroatoms include, but are not limited to nitrogen, oxygen, and sulfur.

“Heteroatom moiety” includes a moiety where the atom by which the moiety is attached is not a carbon. Examples of heteroatom moieties include —N═, —NR_{c}—, —N⁺(O⁻)═, —O—, —S— or —S(O)₂—, wherein R_{c} is further substituent.

“Heteroalkyl” means alkyl, as defined above, provided that one or more of the atoms within the alkyl chain is a heteroatom.

“Heterobicycloalkyl” means bicycloalkyl, as defined above, provided that one or more of the atoms within the ring is a heteroatom. For example hetero(C₉₋₁₂)bicycloalkyl as used in this application includes, but is not limited to, 3-aza-bicyclo[4.1.0]hept-3-yl, 2-aza-bicyclo[3.1.0]hex-2-yl, 3-aza-bicyclo[3.1.0]hex-3-yl, and the like.

“Heterocycloalkylene” means cycloalkylene, as defined above, provided that one or more of the ring member carbon atoms is replaced by a heteroatom.

“Heteroaryl” means a monocyclic or polycyclic aromatic group wherein at least one ring atom is a heteroatom and the remaining ring atoms are carbon. Monocyclic heteroaryl groups include, but are not limited to, cyclic aromatic groups having five or six ring atoms, wherein at least one ring atom is a heteroatom and the remaining ring atoms are carbon. The nitrogen atoms can be optionally quaternized and the sulfur atoms can be optionally oxidized. Heteroaryl groups of this invention include, but are not limited to, those derived from furan, imidazole, isothiazole, isoxazole, oxadiazole, oxazole, 1,2,3-oxadiazole, pyrazine, pyrazole, pyridazine, pyridine, pyrimidine, pyrroline, thiazole, 1,3,4-thiadiazole, triazole and tetrazole. “Heteroaryl” also includes, but is not limited to, bicyclic or tricyclic rings, wherein the heteroaryl ring is fused to one or two rings independently selected from the group consisting of an aryl ring, a cycloalkyl ring, a cycloalkenyl ring, and another monocyclic heteroaryl or heterocycloalkyl ring. These bicyclic or tricyclic heteroaryls include, but are not limited to, those derived from benzo[b]furan, benzo[b]thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2-a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3-c]pyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole and 2(1H)-pyridinone. The bicyclic or tricyclic heteroaryl rings can be attached to the parent molecule through either the heteroaryl group itself or the aryl, cycloalkyl, cycloalkenyl or heterocycloalkyl group to which it is fused. The heteroaryl groups of this invention can be substituted or unsubstituted.

“Heterobicycloaryl” means bicycloaryl, as defined above, provided that one or more of the atoms within the ring is a heteroatom. For example, hetero(C₄₋₁₂)bicycloaryl as used in this application includes, but is not limited to, 2-amino-4-oxo-3,4-dihydropteridin-6-yl, tetrahydroisoquinolinyl, and the like.

“Heterocycloalkyl” means cycloalkyl, as defined above, provided that one or more of the atoms forming the ring is a heteroatom selected, independently from N, O, or S. Non-exclusive examples of heterocycloalkyl include piperidyl, 4-morpholyl, 4-piperazinyl, pyrrolidinyl, perhydropyrrolizinyl, 1,4-diazaperhydroepinyl, 1,3-dioxanyl, 1,4-dioxanyl, tetrazolyl and the like.

“Hydroxy” means the radical —OH.

“IC₅₀” means the molar concentration of an inhibitor that produces 50% inhibition of the target enzyme.

“Iminoketone derivative” means a derivative comprising the moiety —C(NR_{d})—, wherein R_{d} is further substituent and comprises a hydrogen or carbon atom attached to the nitrogen.

“Isomers” mean any compound having an identical molecular formulae but differing in the nature or sequence of bonding of their atoms or in the arrangement of their atoms in space. Isomers that differ in the arrangement of their atoms in space are termed “stereoisomers”. Stereoisomers that are not mirror images of one another are termed “diastereomers” and stereoisomers that are nonsuperimposable mirror images are termed “enantiomers” or sometimes “optical isomers”. A carbon atom bonded to four nonidentical substituents is termed a “chiral center”. A compound with one chiral center has two enantiomeric forms of opposite chirality. A mixture of the two enantiomeric forms is termed a “racemic mixture”. A compound that has more than one chiral center has 2ⁿ⁻¹ enantiomeric pairs, where n is the number of chiral centers. Compounds with more than one chiral center may exist as either an individual diastereomer or as a mixture of diastereomers, termed a “diastereomeric mixture”. When one chiral center is present, a stereoisomer may be characterized by the absolute configuration of the chiral center. Absolute configuration refers to the arrangement in space of the substituents attached to the chiral center. Enantiomers are characterized by the absolute configuration of their chiral centers and described by the R- and S-sequencing rules of Cahn, Ingold and Prelog. Conventions for stereochemical nomenclature, methods for the determination of stereochemistry and the separation of stereoisomers are well known in the art (e.g., see “Advanced Organic Chemistry”, 4th edition, March, Jerry, John Wiley & Sons, New York, 1992).

“Nitro” means the radical —NO₂.

“Oxaalkyl” means an alkyl, as defined above, except where one or more of the carbon atoms forming the alkyl chain are replaced with oxygen atoms (—O—). For example, a (C₁₋₁₀)oxaalkyl refers to a chain comprising between 1 and 10 carbons and one or more oxygen atoms.

“Oxoalkyl” means an alkyl, further substituted with a carbonyl group. In particular, “oxoalkyl” refers to an alkyl, as defined above, wherein one or more of the carbon atoms forming the alkyl chain is substituted with an oxo group (═O). The carbonyl group may be an aldehyde, ketone, ester, amide, acid or acid chloride. For example, a (C₂₋₁₀)oxoalkyl refers to a chain comprising between 2 and 10 carbon atoms wherein one or more of the carbon atoms is substituted with an oxo group to form a carbonyl.

“Oxy” means the radical —O—. It is noted that the oxy radical may be further substituted with a variety of substituents to form different oxy groups including hydroxy, alkoxy, aryloxy, heteroaryloxy or carbonyloxy.

“Pharmaceutically acceptable” means that which is useful in preparing a pharmaceutical composition that is generally safe, non-toxic and neither biologically nor otherwise undesirable and includes that which is acceptable for veterinary use as well as human pharmaceutical use.

“Pharmaceutically acceptable salts” means salts of compounds of the present invention which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as acetic acid, propionic acid, hexanoic acid, heptanoic acid, cyclopentanepropionic acid, glycolic acid, pyruvic acid, lactic acid, malonic acid, succinic acid, malic acid, maleic acid, fumaric acid, tartaric acid, citric acid, benzoic acid, o-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, p-chlorobenzenesulfonic acid, 2-naphthalenesulfonic acid, p-toluenesulfonic acid, camphorsulfonic acid, 4-methylbicyclo[2.2.2]oct-2-ene-1-carboxylic acid, glucoheptonic acid, 4,4′-methylenebis(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid and the like.

Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like.

“Prodrug” means a compound that is convertible in vivo metabolically into a compound represented by the formula (I) (hereinafter also to be abbreviated as compound (I)) to be used for the present invention. The prodrug itself may or may not also have kinase inhibitory activity. For example, a compound comprising a hydroxy group may be administered as an ester that is converted by hydrolysis in vivo to the hydroxy compound. Suitable esters that may be converted in vivo into hydroxy compounds include acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methanesulfonates, ethanesulfonates, benzenesulfonates, p-toluenesulfonates, cyclohexylsulfamates, quinates, esters of amino acids, and the like. Similarly, a compound comprising an amine group may be administered as an amide that is converted by hydrolysis in vivo to the amine compound.

“Protected derivatives” means derivatives of a compound (I) in which a reactive site or sites are blocked with protecting groups. Protected derivatives are useful in the preparation of inhibitors or in themselves may be active as inhibitors. A comprehensive list of suitable protecting groups can be found in T. W. Greene, Protecting Groups in Organic Synthesis, 3rd edition, John Wiley & Sons, Inc. 1999.

“Ring” means a carbocyclic or a heterocyclic system.

“Substituted or unsubstituted” means that a given moiety may consist of only hydrogen substituents through available valencies (unsubstituted) or may further comprise one or more non-hydrogen substituents through available valencies (substituted) that are not otherwise specified by the name of the given moiety. For example, isopropyl is an example of an ethylene moiety that is substituted by —CH₃. In general, a non-hydrogen substituent may be any substituent that may be bound to an atom of the given moiety that is specified to be substituted. Examples of substituents include, but are not limited to, aldehyde, alicyclic, aliphatic, (C₁₋₁₀)alkyl, alkylene, alkylidene, amide, amino, aminoalkyl, aromatic, aryl, bicycloalkyl, bicycloaryl, carbamoyl, carbocyclyl, carboxyl, carbonyl group, cycloalkyl, cycloalkylene, ester, halo, heterobicycloalkyl, heterocycloalkylene, heteroaryl, heterobicycloaryl, heterocycloalkyl, oxo, hydroxy, iminoketone, ketone, nitro, oxaalkyl and oxoalkyl moieties, each of which may optionally also be substituted or unsubstituted. In one particular embodiment, examples of substituents include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀) alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀) alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl. In addition, the substituent is itself optionally substituted by a further substituent. In one particular embodiment, examples of the further substituent include, but are not limited to, hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅) alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₉₋₁₂)bicycloaryl(C₁₋₅)alkyl, (C₃₋₁₂)cycloalkyl, hetero(C₃₋₁₂)cycloalkyl(C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl.

“Sulfinyl” means the radical —SO—. It is noted that the sulfinyl radical may be further substituted with a variety of substituents to form different sulfinyl groups including sulfinic acids, sulfinamides, sulfinyl esters and sulfoxides.

“Sulfonyl” means the radical —SO₂—. It is noted that the sulfonyl radical may be further substituted with a variety of substituents to form different sulfonyl groups including sulfonic acids, sulfonamides, sulfonate esters and sulfones.

“Therapeutically effective amount” means that amount which, when administered to an animal for treating a disease, is sufficient to effect such treatment for the disease.

“Thioalkyl” means an alkyl, as defined above, except where one or more of the carbon atoms forming the alkyl chain are replaced with sulfur atoms (—S—). For example, a (C₁₋₁₀ thioalkyl refers to a chain comprising between 1 and 10 carbons and one or more sulfur atoms.

“Thiocarbonyl” means the radical —CS—. It is noted that the thiocarbonyl radical may be further substituted with a variety of substituents to form different thiocarbonyl groups including thioacids, thioamides, thioesters and thioketones.

“Treatment” or “treating” means any administration of a compound (I) to be used for the present invention and includes:

(1) preventing a disease from occurring in an animal which may be predisposed to the disease but does not yet experience or display the pathology or symptomatology of the disease,
(2) inhibiting a disease in an animal that is experiencing or displaying the pathology or symptomatology of the disease (i.e., arresting further development of the pathology and/or symptomatology), and
(3) ameliorating a disease in an animal that is experiencing or displaying the pathology or symptomatology of the disease (i.e., reversing the pathology and/or symptomatology).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₑR_{f}R_{g} where Rₑ, R_{f} and R_{g} may each independently be hydrogen or any other substituent where the atom attached to the carbon is a heteroatom or cyano. That is, for example, CF₃, CH₂OH and CH₂CN are all included in C₁ alkyls.

### Compound (I)

The pharmaceutical composition of the present invention comprises a compound represented by the formula (I) (compound (I)):

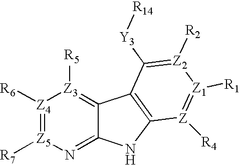

wherein
Z, Z₁, Z₂, Z₃, Z₄ and Z₅ are each independently selected from the group consisting of C and N;
R₁ is —Y₁—R₁₂, or R₁ is absent when Z₁ is N;
R₂ is —Y₂—R₁₃, or R₂ is absent when Z₂ is N, or R₁ and
R₂ are taken together to form a substituted or unsubstituted ring;
Y₁, Y₂ and Y₃ are each independently absent or a linker providing 1 or 2 atom separation between R₁₂, R₁₃ or R₁₄ and the ring to which Y₁, Y₂ or Y₃ is attached, wherein the atoms of the linker providing the separation are selected from the group consisting of carbon, oxygen, nitrogen and sulfur;
R₄ is selected from the group consisting of hydrogen, halo, nitro, cyano, thio, oxy, hydroxy, carbonyloxy, alkoxy, carbonyl, amino, (C₁₋₅)alkylamino, (C₁₋₅)alkyl, halo(C₁₋₅)alkyl, carbonyl(C₁₋₃)alkyl, sulfonyl(C₁₋₃)alkyl, amino(C₁₋₅)alkyl, aryl(C₁₋₅)alkyl, heteroaryl(C₁₋₅)alkyl, (C₃₋₆)cycloalkyl and hetero(C₃₋₆)cycloalkyl, each substituted or unsubstituted, with the proviso that R₄ is absent when the atom to which it is bound is N;
R₅ and R₆ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀ alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, with the proviso that each of R₅ and R₆ is absent when the atom to which it is bound is N;
R₇ is selected from the group consisting of hydrogen, halo, hydroxy, alkoxy, amino and (C₁₋₅)alkyl, each substituted or unsubstituted, with the proviso that R₇ is absent when the atom to which it is bound is N;

R₁₂ and R₁₃ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₂ and R₁₃ are taken together to form a substituted or unsubstituted ring; and
R₁₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃) alkyl, sulfonyl(C₁₋₃)alkyl, sulfinyl(C₁₋₃)alkyl, amino(C₁₋₁₀) alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In the above formula, (a) —Y₃—R₁₄ is not H when Z, Z₁, Z₂, Z₃ and Z₅ are all C; R₅ is a substituted amino group; and R₂ is methoxy or R₇ is methyl or amino; and (b) R₁₄ is not 3-chlorophenyl when R₁, R₅, R₆ and R₇ are each H; Z and Z₂ are each N; R₂ and R₄ are absent; Z₁, Z₃, Z₄ and Z₅ are all C; and Y₃ is NH.

In one embodiment, the compound (I) to be used for the present invention is represented by the formula (Ia):

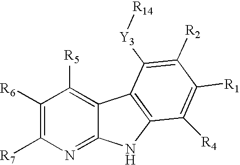

wherein each symbol is as defined above.

In another embodiment, the compound (I) to be used for the present invention is represented by the formula (Ib):

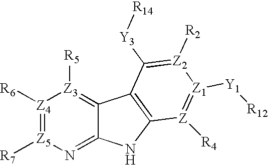

wherein each symbol is as defined above.

In still another embodiment, the compound (I) to be used for the present invention is represented by the formula (Ic):

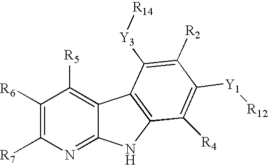

wherein each symbol is as defined above.

In yet another embodiment, the compound (I) to be used for the present invention is represented by the formula (Id):

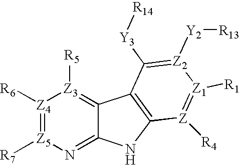

wherein each symbol is as defined above.

In a further embodiment, the compound (I) to be used for the present invention is represented by the formula (Ie):

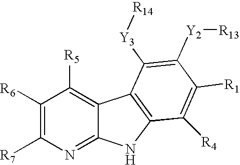

wherein each symbol is as defined above.

In a still further embodiment, the compound (I) to be used for the present invention is represented by the formula (If):

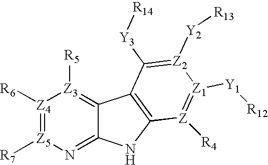

wherein each symbol is as defined above.

In one variation of the above embodiment, —Y₁—R₁₂ is absent when Z₁ is N and —Y₂—R₁₃ is absent when Z₂ is N.

In another embodiment, the compound (I) to be used for the present invention is represented by the formula (Ig):

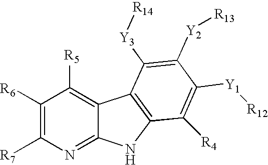

wherein each symbol is as defined above.

In still another embodiment, the compound (I) to be used for the present invention is represented by the formula (Ih):

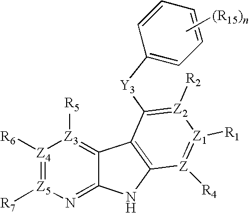

wherein
n is selected from the group consisting of 0, 1, 2, 3, 4 and 5;
each R₁₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₁₅ are taken together to form a substituted or unsubstituted ring; and other symbols are as defined above.

In one variation of the above embodiment, R₁₅ is not 3-chloro when n is 1; R₁, R₅, R₆ and R₇ are each H; Z and Z₂ are each N; R₂ and R₄ are absent; Z₁, Z₃, Z₄ and Z₅ are all C; and Y₃ is NH.

In yet another embodiment, the compound (I) to be used for the present invention is represented by the formula (Ii):

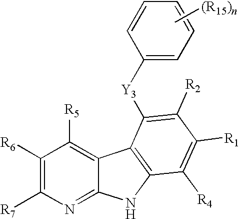

wherein
n is selected from the group consisting of 0, 1, 2, 3, 4 and 5;
each R₁₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂) bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₁₅ are taken together to form a substituted or unsubstituted ring; and other symbols are as defined above.

In a further embodiment, the compound (I) to be used for the present invention is represented by the formula (Ij):

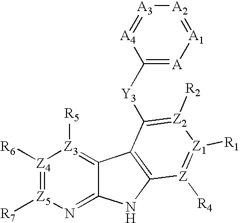

wherein
A, A₁, A₂, A₃ and A₄ are each independently selected from the group consisting of CR₂₅ and N;
each R₂₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring; and other symbols are as defined above.

In one variation of the above embodiment, A₁ is not CCl when A, A₂, A₃ and A₄ are each CH; R₁, R₅, R₆ and R₇ are each H; Z and Z₂ are each N; R₂ and R₄ are absent; Z₁, Z₃, Z₄ and Z₅ are all C; and Y₃ is NH.

In still a further embodiment, the compound (I) to be used for the present invention is represented by the formula (Ik):

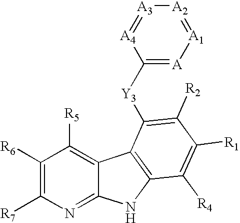

wherein
A, A₁, A₂, A₃ and A₄ are each independently selected from the group consisting of CR₂₅ and N; and
each R₂₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring; and other symbols are as defined above.

In yet a further embodiment, the compound (I) to be used for the present invention is represented by the formula (Il)

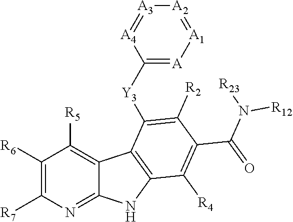

wherein
A, A₁, A₂, A₃ and A₄ are each independently selected from the group consisting of CR₂₅ and N;

R₂₃ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C_{3-3.2})cycloalkyl, aryl and heteroaryl, each substituted or unsubstituted, or R₂₃ and R₁₂ are taken together to form a substituted or unsubstituted ring;
each R₂₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring; and other symbols are as defined above.

In another embodiment, the compound (I) to be used for the present invention is represented by the formula (Im):

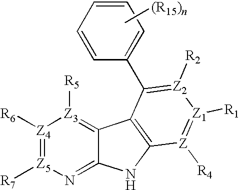

wherein
n is selected from the group consisting of 0, 1, 2, 3, 4 and 5;
each R₁₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₁₅ are taken together to form a substituted or unsubstituted ring; and other symbols are as defined above.

In still another embodiment, the compound (I) to be used for the present invention is represented by the formula (In):

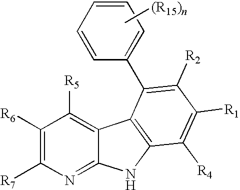

wherein
n is selected from the group consisting of 0, 1, 2, 3, 4 and 5;
each R₁₅ is independently selected from the group consisting of hydrogen, halo, nitro, cyano, thio, oxy, hydroxy, alkoxy, aryloxy, heteroaryloxy, carbonyl, amino, alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₁₅ are taken together to form a substituted or unsubstituted ring; and other symbols are as defined above.

In yet another embodiment, the compound (I) to be used for the present invention is represented by the formula (Io):

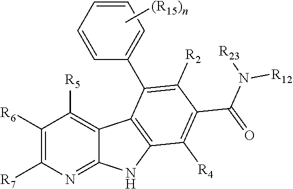

wherein
n is selected from the group consisting of 0, 1, 2, 3, 4 and 5;
each R₁₅ is independently selected from the group consisting of hydrogen, halo, nitro, cyano, thio, oxy, hydroxy, alkoxy, aryloxy, heteroaryloxy, carbonyl, amino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₃₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₁₅ are taken together to form a substituted or unsubstituted ring;
R₂₃ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅) alkyl, (C₃₋₁₂)cycloalkyl, hetero(C₃₋₁₂)cycloalkyl, aryl and heteroaryl, each substituted or unsubstituted, or R₂₃ and R₁₂ are taken together to form a substituted or unsubstituted ring; and other symbols are as defined above.

In a further embodiment, the compound (I) to be used for the present invention is represented by the formula (Ip):

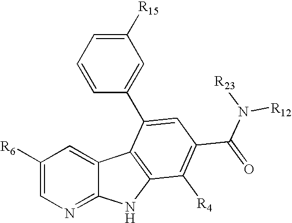

wherein

R₁₅ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero (C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;

R₂₃ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₁₂ are taken together to form a substituted or unsubstituted ring; and other symbols are as defined above.

In still a further embodiment, the compound (I) to be used for the present invention is represented by the formula (Iq)

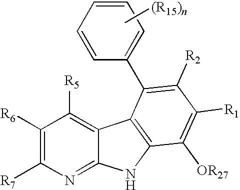

wherein
n is selected from the group consisting of 0, 1, 2, 3, 4 and 5;
each R₁₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₁₅ are taken together to form a substituted or unsubstituted ring;
R₂₇ is selected from the group consisting of hydrogen, halo, nitro, cyano, thio, oxy, hydroxy, carbonyloxy, alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀) alkyl, (C₁₋₁₀)azaalkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂) bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and other symbols are as defined above.

In yet a further embodiment, the compound (I) to be used for the present invention is represented by the formula (Ir):

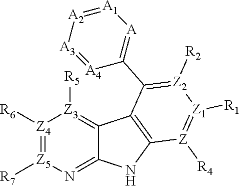

wherein
A, A₁, A₂, A₃ and A₄ are each independently selected from the group consisting of CR₂₅ and N;
each R₂₅ is independently selected from the group consisting of hydrogen, halo, nitro, cyano, thio, oxy, hydroxy, alkoxy, aryloxy, heteroaryloxy, carbonyl, amino, alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring; and other symbols are as defined above.

In another embodiment, the compound (I) to be used for the present invention is represented by the formula (Is):

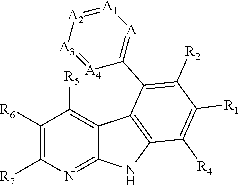

wherein
A, A₁, A₂, A₃ and A₄ are each independently selected from the group consisting of CR₂₅ and N;
each R₂₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring; and other symbols are as defined above.

In still another embodiment, the compound (I) to be used for the present invention is represented by the formula (It):

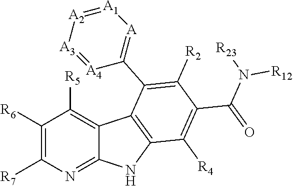

wherein
A, A₁, A₂, A₃ and A₄ are each independently selected from the group consisting of CR₂₅ and N;
R₂₃ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₁₂ are taken together to form a substituted or unsubstituted ring;
each R₂₅ is independently selected from the group consisting of hydrogen, halo, nitro, cyano, thio, oxy, hydroxy, alkoxy, aryloxy, heteroaryloxy, carbonyl, amino, alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring; and other symbols are as defined above.

In yet another embodiment, the compound (I) to be used for the present invention is represented by the formula (Iu):

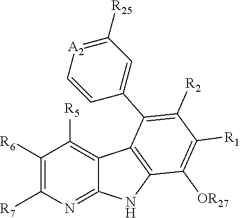

wherein
A₂ is selected from the group consisting of CR₂₅ and N;
each R₂₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero (C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring;

R₂₇ is selected from the group consisting of hydrogen, halo, nitro, cyano, thio, oxy, hydroxy, carbonyloxy, (C₁₋₁₀) alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀) aryl(C₁₋₅)alkyl, (C₉₋₁₂)bicycloaryl(C₁₋₅)alkyl, hetero(C₈₋₁₂) bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and other symbols are as defined above.

In a further embodiment, the compound (I) to be used for the present invention is represented by the formula (Iv):

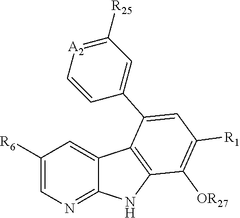

wherein
A₂ is selected from the group consisting of CR₂₅ and N;
each R₂₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a ring;
R₂₇ is selected from the group consisting of hydrogen, halo, nitro, cyano, thio, oxy, hydroxy, carbonyloxy, alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀) alkyl, (C₁₋₁₀)azaalkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀) aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and other symbols are as defined above.

In still a further embodiment, the compound (I) to be used for the present invention is represented by the formula (Iw):

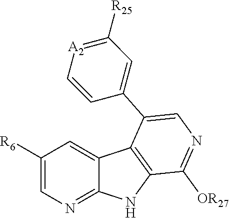

wherein
A₂ is selected from the group consisting of CR₂₅ and N;
each R₂₅ is independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₈₋₁₂)bicycloaryl and hetero(C₄₋₁₂)bicycloaryl, each substituted or unsubstituted, or any two R₂₅ are taken together to form a substituted or unsubstituted ring;
R₂₇ is selected from the group consisting of hydrogen, halo, nitro, cyano, thio, oxy, hydroxy, carbonyloxy, (C₁₋₁₀) alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀) aryl(C₁₋₅)alkyl, (C₉₋₁₂)bicycloaryl(C₁₋₅)alkyl, hetero(C₈₋₁₂) bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and other symbols are as defined above.

In yet a further embodiment, the compound (I) to be used for the present invention is represented by the formula (Ix):

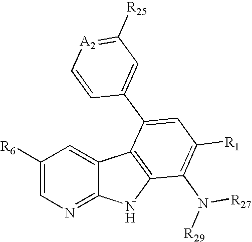

wherein
A₂ is selected from the group consisting of CR₂₅ and N;
each R₂₅ is independently selected from the group consisting of hydrogen, halo, nitro, cyano, thio, oxy, hydroxy, alkoxy, aryloxy, heteroaryloxy, carbonyl, amino, alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or any two R₂₅ are taken together to form a substituted or unsubstituted ring;
R₂₇ and R₂₉ are each independently selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀) alkyl, sulfinyl(C₁₋₁₀)alkyl, (C₁₋₁₀)azaalkyl, (C₁₋₁₀)oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₈₋₁₂)bicycloaryl(C₁₋₅) alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted, or R₂₇ and R₂₉ are taken together to form a substituted or unsubstituted ring; and other symbols are as defined above.

In another embodiment, the compound (I) to be used for the present invention is represented by the formula (Iy):

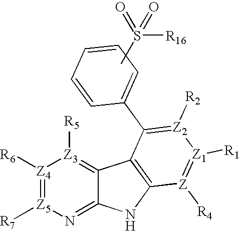

wherein
R₁₆ is selected from the group consisting of amino, (C₁₋₁₀)alkylamino, (C₁₋₁₀)alkyl, halo(C₁₋₁₀)alkyl, hydroxy(C₁₋₅)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted; and other symbols are as defined above.

In still another embodiment, the compound (I) to be used for the present invention is represented by the formula (Iz):

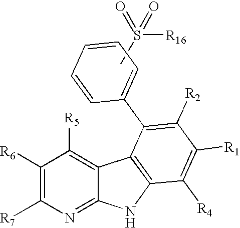

wherein
R₁₆ is selected from the group consisting of amino, (C₁₋₁₀)alkylamino, (C₁₋₁₀)alkyl, halo(C₁₋₁₀)alkyl, hydroxy(C₁₋₅)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, (C₃₋₁₂)cycloalkyl(C₁₋₅)alkyl, hetero(C₃₋₁₂)cycloalkyl(C₁₋₅) alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted; and other symbols are as defined above.

In yet another embodiment, the compound (I) to be used for the present invention is represented by the formula (Iaa):

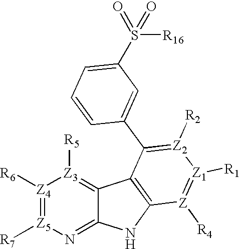

wherein
R₁₆ is selected from the group consisting of amino, (C₁₋₁₀)alkylamino, (C₁₋₁₀)alkyl, halo(C₁₋₁₀)alkyl, hydroxy(C₁₋₅)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted; and other symbols are as defined above.

In a further embodiment, the compound (I) to be used for the present invention is represented by the formula (Ibb):

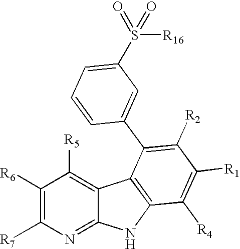

wherein
R₁₆ is selected from the group consisting of amino, (C₁₋₁₀)alkylamino, (C₁₋₁₀)alkyl, halo(C₁₋₁₀)alkyl, hydroxy(C₁₋₅)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, (C₃₋₁₂)cycloalkyl(C₁₋₅)alkyl, hetero(C₃₋₁₂)cycloalkyl(C₁—) alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted; and other symbols are as defined above.

In still a further embodiment, the compound (I) to be used for the present invention is represented by the formula (Icc):

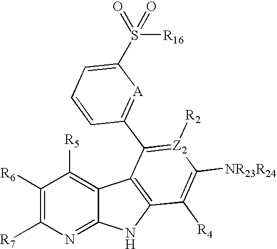

wherein
A is selected from the group consisting of CR₂₅ and N;
R₁₆ is selected from the group consisting of amino, (C₁₋₁₀)alkylamino, (C₁₋₁₀)alkyl, halo(C₁₋₁₀)alkyl, hydroxy(C₁₋₅) alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, (C₃₋₁₂)cycloalkyl(C₁₋₅)alkyl, hetero(C₃₋₁₂)cycloalkyl(C₁₋₅) alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted;
R₂₃ and R₂₄ are each independently selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃) alkyl, sulfonyl(C₁₋₃)alkyl, sulfinyl(C₁₋₃)alkyl, amino(C₁₋₁₀) alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₂₄ are taken together to form a substituted or unsubstituted ring; R₂₅ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃) alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and other symbols are as defined above.

In yet a further embodiment, the compound (I) to be used for the present invention is represented by the formula (Idd):

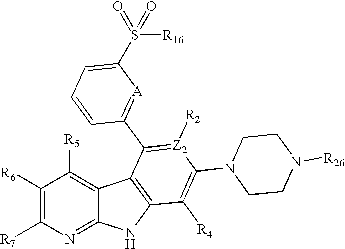

wherein
A is selected from the group consisting of CR₂₅ and N;
R₁₆ is selected from the group consisting of amino, (C₁₋₁₀)alkylamino, (C₁₋₁₀)alkyl, halo(C₁₋₁₀)alkyl, hydroxy(C₁₋₅) alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, (C₃₋₁₂)cycloalkyl(C₁₋₅)alkyl, hetero(C₃₋₁₂)cycloalkyl(C₁₋₅) alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted;
R₂₅ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃) alkyl, sulfonyl(C₁₋₃)alkyl, sulfinyl(C₁₋₃)alkyl, amino(C₁₋₁₀) alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₂₆ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted; and other symbols are as defined above.

In another embodiment, the compound (I) to be used for the present invention is represented by the formula (Iee):

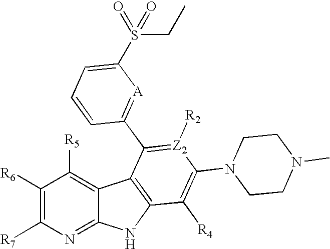

wherein
A is selected from the group consisting of CR₂₅ and N;
R₂₅ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃) alkyl, sulfonyl(C₁₋₃)alkyl, sulfinyl(C₁₋₃)alkyl, amino(C₁₋₁₀) alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and other symbols are as defined above.

In still another embodiment, the compound (I) to be used for the present invention is represented by the formula (Iff):

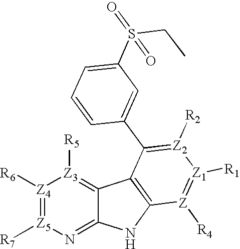

wherein each symbol is as defined above.

In yet another embodiment, the compound (I) to be used for the present invention is represented by the formula (Igg):

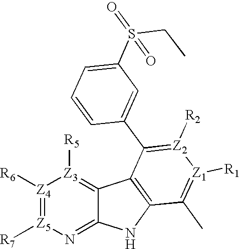

wherein each symbol is as defined above.

In a further embodiment, the compound (I) to be used for the present invention is represented by the formula (Ihh):

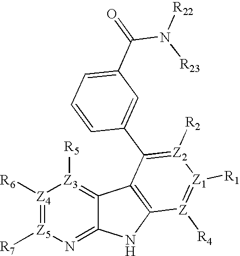

wherein
R₂₂ is selected from the group consisting of hydrogen, halo, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃) alkyl, sulfonyl(C₁₋₃)alkyl, sulfinyl(C₁₋₃)alkyl, amino(C₁₋₁₀) alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted;
R₂₃ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₂₂ are taken together to form a substituted or unsubstituted ring; and other symbols are as defined above.

The compound (I) to be used for the present invention can be produced according to Examples of WO2007/044779 and US2007/0117816.

In one variation of each compound of the above embodiments and variations, A is CR₂₅. In another variation of each compound of the above embodiments and variations, A₁ is CR₂₅. In still another variation of each compound of the above embodiments and variations, A₂ is CR₂₅. In yet another variation of each compound of the above embodiments and variations, A₃ is CR₂₅. In a further variation of each compound of the above embodiments and variations, A₄ is CR₂₅.

In another variation of each compound of the above embodiments and variations, Y₁ is selected from the group consisting of —CH₂—, —NH—, —O— and —S—.

In still another variation of each compound of the above embodiments and variations, Y₁ is selected from the group consisting of —O—, —(CR₁₉R₂₀)ₘ—, —S— and —S—CH₂—, wherein m is selected from the group consisting of 0, 1, 2, 3, 4 and 5; R₁₉ and R₂₀ are each independently selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₂ and any one of R₁₉ are taken together to form a substituted or unsubstituted ring; and R₂₁ is selected from the group consisting of hydrogen, carbonyloxy, alkoxy, aryloxy, heteroaryloxy, carbonyl, oxycarbonyl, (C₁₋₁₀)alkylamino, sulfonamido, imino, sulfonyl, sulfinyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂) cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₂₁ and R₁₂ are taken together to form a substituted or unsubstituted ring.

In yet another variation of each compound of the above embodiments and variations, Y₁ is —C(O)—NR₂₃—, wherein R₂₃ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃) alkyl, sulfonyl(C₁₋₃)alkyl, sulfinyl(C₁₋₃)alkyl, amino(C₁₋₁₀) alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₁₂ are taken together to form a substituted or unsubstituted ring.

In a further variation of each compound of the above embodiments and variations, Y₁ is —C(O)—O—.

In still a further variation of each compound of the above embodiments and variations, Y₁ is —NR₂₃—C(O)—, wherein R₂₃ is selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₁₂ are taken together to form a substituted or unsubstituted ring.

In yet a further variation of each compound of the above embodiments and variations, Y₂ is selected from the group consisting of —CH₂—, —NH—, —O— and —S—.

In another variation of each compound of the above embodiments and variations, Y₂ is selected from the group consisting of —O—, —(CR₁₉R₂₀)ₘ—, —NR₂₁—, —S— and —S—CH₂—, wherein m is selected from the group consisting of 0, 1, 2, 3, 4 and 5; R₁₉ and R₂₀ are each independently selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₃ and any one of R₁₉ are taken together to form a substituted or unsubstituted ring; and R₂₁ is selected from the group consisting of hydrogen, carbonyloxy, alkoxy, aryloxy, heteroaryloxy, carbonyl, oxycarbonyl, (C₁₋₁₀)alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₂₁ and R₁₃ are taken together to form a substituted or unsubstituted ring.

In still another variation of each compound of the above embodiments and variations, Y₃ is selected from the group consisting of —CH₂—, —NH—, —O— and —S—.

In yet another variation of each compound of the above embodiments and variations, Y₃ is selected from the group consisting of —O—, —(CR₁₉R₂₀)ₘ—, —NR₂₁—, —S— and —S—CH₂—, wherein m is selected from the group consisting of 0, 1, 2, 3, 4 and 5; R₁₉ and R₂₀ are each independently selected from the group consisting of hydrogen, halo, cyano, thio, oxy, hydroxy, carbonyloxy, alkoxy, aryloxy, heteroaryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and R₂₁ is selected from the group consisting of hydrogen, carbonyloxy, alkoxy, aryloxy, heteroaryloxy, carbonyl, oxycarbonyl, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted.

In a further variation of each compound of the above embodiments and variations, Y₃ is absent.

In still a further variation of each compound of the above embodiments and variations, —Y₃—R₁₄ is selected from the group consisting of aryl, heteroaryl, (C₉₋₁₂)bicycloaryl and hetero(C₄₋₁₂)bicycloaryl, each substituted or unsubstituted.

In yet a further variation of each compound of the above embodiments and variations, Z is N. In another variation of each compound of the above embodiments and variations, Z₁ is N. In still another variation of each compound of the above embodiments and variations, Z₂ is N. In yet another variation of each compound of the above embodiments and variations, Z₃ is N. In a further variation of each compound of the above embodiments and variations, Z₄ is N. In still a further variation of each compound of the above embodiments and variations, Z₅ is N. In yet a further variation of each compound of the above embodiments and variations, Z, Z₂, Z₃, Z₄ and Z₅ are each C. In another variation of each compound of the above embodiments and variations, Z, Z₁, Z₂, Z₃, Z₄ and Z₅ are each C.

In still another variation of each compound of the above embodiments and variations, R₁ is selected from the group consisting of hydrogen, halo, amino, alkoxy, carbonyloxy, aminocarbonyl, sulfonyl, carbonylamino, sulfonylamino, (C₁₋₁₀)alkyl, hetero(C₃₋₁₂)cycloalkyl and aryl, each substituted or unsubstituted. In yet another variation of each compound of the above embodiments and variations, R₁ is a substituted or unsubstituted piperadinyl. In a further variation of each compound of the above embodiments and variations, R₁ is a substituted or unsubstituted 1-methyl(piperadin-4-yl).

In still a further variation of each compound of the above embodiments and variations, R₂ is selected from the group consisting of hydrogen, halo, amino, alkoxy, (C₁₋₁₀)alkyl, hetero(C₃₋₁₂)cycloalkyl and aryl, each substituted or unsubstituted. In yet a further variation of each compound of the above embodiments and variations, R₂ is hydrogen.

In another variation of each compound of the above embodiments and variations, R₄ is selected from the group consisting of hydrogen, halo and substituted or unsubstituted (C₁₋₅)alkyl. In still another variation of each compound of the above embodiments and variations, R₄ is methyl. In yet another variation of each compound of the above embodiments and variations, R₄ is trifluoromethyl. In a further variation of each compound of the above embodiments and variations, R₄ is substituted or unsubstituted oxaalkyl. In still a further variation of each compound of the above embodiments and variations, R₄ is a substituted or unsubstituted alkoxy. In yet a further variation of each compound of the above embodiments and variations, R₄ is substituted or unsubstituted aryloxy.

In another variation of each compound of the above embodiments and variations, R₄ is —Y₄—R₂₇, wherein Y₄ is absent or a linker providing 1 or 2 atom separation between R₂₇ and the ring to which Y₄ is attached; and R₂₇ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀) aryloxy, carbonyl, oxycarbonyl, aminocarbonyl, amino, (C₁₋₁₀) alkylamino, sulfonamido, imino, sulfonyl, sulfinyl, (C₁₋₁₀) alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, (C₁₋₁₀)oxaalkyl, oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀) aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C_{4-3.2})bicycloaryl, each substituted or unsubstituted. In one variation, Y₄ is selected from the group consisting of —CH₂—, —NH—, —O— and —S—. In another variation, Y₄ is absent.

In still another variation of each compound of the above embodiments and variations, R₄ is —OR₂₇, wherein R₂₇ is selected from the group consisting of hydrogen, halo, nitro, cyano, thio, oxy, hydroxy, carbonyloxy, (C₁₋₁₀)alkoxy, (C₄₋₁₂) aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀) alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, oxaalkyl, (C₁₋₁₀)oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅)alkyl, (C₉₋₁₂)bicycloaryl(C₁₋₅)alkyl, hetero(C₉₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero C₁₋₁₀)aryl, (C₉₋₁₂)bicycloaryl and hetero(C₄₋₁₂)bicycloaryl, each substituted or unsubstituted.

In yet another variation of each compound of the above embodiments and variations, R₄ is —SR₂₇, wherein R₂₇ is selected from the group consisting of hydrogen, halo, nitro, cyano, thio, oxy, hydroxy, carbonyloxy, (C₁₋₁₀)alkoxy, (C₄₋₁₂)aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀) alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, (C₁₋₁₀)oxaalkyl, oxoalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀) aryl(C₁₋₅)alkyl, (C₉₋₁₂)bicycloaryl(C₁₋₅)alkyl, hetero(C₈₋₁₂) bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted.

In a further variation of each compound of the above embodiments and variations, R₄ is —NR₂₈—R₂₇, wherein R₂₇ is selected from the group consisting of hydrogen, halo, nitro, cyano, thio, oxy, hydroxy, carbonyloxy, (C₁₋₁₀)alkoxy, (C₄₋₁₂) aryloxy, hetero(C₁₋₁₀)aryloxy, carbonyl, oxycarbonyl, aminocarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, oxaalkyl, (C₁₋₁₀)oxoalkyl, imino(C₁₋₁₀)alkyl, (C₃₋₁₂)cycloalkyl(C₁₋₃)alkyl, hetero(C₃₋₁₂)cycloalkyl(C₁₋₁₀)alkyl, aryl(C₁₋₁₀)alkyl, hetero aryl(C₁₋₅)alkyl, (C₉₋₁₂)bicycloaryl(C₁₋₅)alkyl, hetero(C₈₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted; and R₂₈ is selected from the group consisting of hydrogen, oxy, hydroxy, carbonyloxy, (C₁₋₁₀)alkoxy, (C₄₋₁₂)aryloxy, hetero(C₁₋₁₀)aryloxy, carbonyl, oxycarbonyl, amino, (C₁₋₁₀)alkylamino, sulfonamido, imino, sulfonyl, sulfinyl, (C₁₋₁₀)alkyl, halo(C₁₋₁₀)alkyl, hydroxy(C₁₋₁₀)alkyl, carbonyl(C₁₋₁₀)alkyl, thiocarbonyl(C₁₋₁₀)alkyl, sulfonyl(C₁₋₁₀)alkyl, sulfinyl(C₁₋₁₀)alkyl, (C₁₋₁₀)azaalkyl, imino(C₁₋₁₀)alkyl, (C₃₋₁₂)cycloalkyl(C₁₋₅)alkyl, hetero(C₃₋₁₂)cycloalkyl(C₁₋₁₀)alkyl, aryl(C₁₋₁₀)alkyl, hetero(C₁₋₁₀)aryl(C₁₋₅) alkyl, (C₉₋₁₂)bicycloaryl(C₁₋₅)alkyl, hetero(C₃₋₁₂)bicycloaryl(C₁₋₅)alkyl, hetero(C₁₋₁₀)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, (C₄₋₁₂)aryl, hetero(C₁₋₁₀)aryl, (C₉₋₁₂)bicycloaryl and hetero(C₄₋₁₂)bicycloaryl, each substituted or unsubstituted. In one variation, R₂₈ is selected from the group consisting of hydrogen and a substituted or unsubstituted (C₁₋₅)alkyl.

In still a further variation of each compound of the above embodiments and variations, R₅ is selected from the group consisting of hydrogen, halo and substituted or unsubstituted (C₁₋₅)alkyl. In yet a further variation of each compound of the above embodiments and variations, R₅ is hydrogen.

In another variation of each compound of the above embodiments and variations, R₆ is selected from the group consisting of hydrogen, halo, amino, carbonyl, alkoxy and (C₁₋₅)alkyl, each substituted or unsubstituted. In a further variation of each compound of the above embodiments and variations, R₆ is a substituted or unsubstituted (C₁₋₅)alkyl. In still another variation of each compound of the above embodiments and variations, R₆ is halo. In yet another variation of each compound of the above embodiments and variations, R₆ is selected from the group consisting of methyl, ethyl, isopropyl and cyclopropyl, each substituted or unsubstituted.

In a further variation of each compound of the above embodiments and variations, R₇ is selected from the group consisting of hydrogen, hydroxy, amino and (C₁₋₅)alkyl, each substituted or unsubstituted. In still a further variation of each compound of the above embodiments and variations, R₇ is hydrogen.

In yet a further variation of each compound of the above embodiments and variations, R₁₂ is selected from the group consisting of hydrogen, halo, amino, alkoxy, carbonyloxy, aminocarbonyl, sulfonyl, carbonylamino, sulfonylamino, (C₁₋₁₀)alkyl, hetero(C₃₋₁₂)cycloalkyl and aryl, each substituted or unsubstituted.

In another variation of each compound of the above embodiments and variations, R₁₃ is selected from the group consisting of hydrogen, halo, amino, alkoxy, carbonyloxy, aminocarbonyl, sulfonyl, carbonylamino, sulfonylamino, (C₁₋₁₀)alkyl, hetero(C₃₋₁₂)cycloalkyl and aryl, each substituted or unsubstituted.

In still another variation of each compound of the above embodiments and variations, R₁₄ is selected from the group consisting of halo, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂) cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted. In yet another variation of each compound of the above embodiments and variations, R₁₄ is selected from the group consisting of (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted. In a further variation of each compound of the above embodiments and variations, R₁₄ is selected from the group consisting of aryl and heteroaryl, each substituted with a substituent selected from the group consisting of halo, carbonyl, (C₁₋₅)alkyl, alkoxy, aminocarbonyl, amino and sulfonyl, each substituted or unsubstituted.

In still a further variation of each compound of the above embodiments and variations, R₁₅ is selected from the group consisting of (C₁₋₁₀)alkyl, —OR₂₂, —C(O)—R₂₂, —NR₂₃—C(O)—R₂₂, —C(O)—NR₂₃—R₂₂, —SO₂—R₂₂, —NR₂₃—SO₂—R₂₂ and —SO₂—NR₂₃R₂₄, wherein R₂₂ is selected from the group consisting of hydrogen, halo, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and R₂₃ and R₂₄ are each independently selected from the group consisting of hydrogen, carbonyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₂₄ are taken together to form a substituted or unsubstituted ring.

In yet a further variation of each compound of the above embodiments and variations, R₁₆ is —NR₂₃—C(O)—R₂₂, wherein R₂₂ is selected from the group consisting of hydrogen, halo, nitro, cyano, thio, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted; and R₂₃ is selected from the group consisting of hydrogen, carbonyl, (C₁₋₁₀)alkyl, halo(C₁₋₁₀) alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)cycloalkyl, hetero(C₃₋₁₂)cycloalkyl, aryl and heteroaryl, each substituted or unsubstituted, or R₂₃ and R₂₄ are taken together to form a substituted or unsubstituted ring.

In another variation of each compound of the above embodiments and variations, R₂₂ is a substituted or unsubstituted (C₃₋₆)cycloalkyl. In still another variation of each compound of the above embodiments and variations, R₂₂ is substituted or unsubstituted cyclopropyl.

In yet another variation of each compound of the above embodiments and variations, R₂₃ and R₂₄ are taken together to form a carbocyclic or heterocyclic (C₅₋₁₀) ring. In a further variation of each compound of the above embodiments and variations, R₂₃ and R₂₄ are taken together to form a substituted or unsubstituted piperazine.

In still a further variation of each compound of the above embodiments and variations, R₂₃ is hydrogen.

In yet a further variation of each compound of the above embodiments and variations, R₂₅ is hydrogen.

In another variation of each compound of the above embodiments and variations, R₂₇ is a substituted or unsubstituted heterocycloalkyl(C₁₋₃)alkyl. In still another variation of each compound of the above embodiments and variations, R₂₇ is a substituted or unsubstituted piperadinyl(C₁₋₃)alkyl. In yet another variation of each compound of the above embodiments and variations, R₂₇ is a substituted or unsubstituted 1-methyl(piperadin-4-yl)(C₁₋₃)alkyl. In a further variation of each compound of the above embodiments and variations, R₂₇ is a substituted or unsubstituted 1-methyl(piperadin-4-yl)methyl. In still a further variation of each compound of the above embodiments and variations, R₂₇ is substituted or unsubstituted amino(C₁₋₅)alkyl. In yet a further variation of each compound of the above embodiments and variations, R₂₇ is substituted or unsubstituted dimethylaminopropyl.

As the compound (I) to be used for the present invention, 5-(3-(ethylsulfonyl)phenyl)-3,8-dimethyl-N-(1-methylpiperidin-4-yl)-9H-pyrido[2,3-b]indole-7-carboxamide or a salt thereof is preferable, and 5-(3-(ethylsulfonyl)phenyl)-3,8-dimethyl-N-(1-methylpiperidin-4-yl)-9H-pyrido[2,3-b]indole-7-carboxamide hydrochloride is particularly preferable.

### Salts, Hydrates, and Prodrugs of Kinase Inhibitors

It should be recognized that compound (I) to be used for the present invention may be present and optionally used in the form of salts, hydrates and prodrugs that are converted in vivo into the compounds.

When the compound (I) to be used for the present invention possesses a free base form, a pharmaceutically acceptable acid addition salt thereof can be prepared by reacting the free base form of the compound with a pharmaceutically acceptable inorganic or organic acid. Examples of such acids are hydrohalides such as hydrochloride, hydrobromide, hydroiodide; other mineral acids and their corresponding salts such as sulfate, nitrate, phosphate, etc.; and alkyl and monoarylsulfonates such as ethanesulfonate, toluenesulfonate and benzenesulfonate; and other organic acids and their corresponding salts such as acetate, tartrate, maleate, succinate, citrate, benzoate, salicylate and ascorbate. Further acid addition salts of the compound (I) include, but are not limited to: adipate, alginate, arginate, aspartate, bisulfate, bisulfite, bromide, butyrate, camphorate, camphorsulfonate, caprylate, chloride, chlorobenzoate, cyclopentanepropionate, digluconate, dihydrogenphosphate, dinitrobenzoate, dodecylsulfate, fumarate, galacterate (from mucic acid), galacturonate, glucoheptaoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate and phthalate.

When the compound (I) to be used for the present invention possesses a free acid form, a pharmaceutically acceptable base addition salt thereof can be prepared by reacting the free acid form of the compound with a pharmaceutically acceptable inorganic or organic base. Examples of such bases are alkali metal hydroxides including potassium, sodium and lithium hydroxides; alkaline earth metal hydroxides such as barium and calcium hydroxides; alkali metal alkoxides, e.g. potassium ethanolate and sodium propanolate; and various organic bases such as ammonium hydroxide, piperidine, diethanolamine and N-methylglutamine. Also included are the aluminum salts of the compound (I). Further base salts of the compound (I) include, but are not limited to: copper, ferric, ferrous, lithium, magnesium, manganic, manganous, potassium, sodium and zinc salts. Organic base salts include, but are not limited to, salts of primary, secondary and tertiary amines, substituted amines including naturally occurring substituted amines, cyclic amines and basic ion exchange resins, e.g., arginine, betaine, caffeine, chloroprocaine, choline, N,N′-dibenzylethylenediamine(benzathine), dicyclohexylamine, diethanolamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine and tris-(hydroxymethyl)-methylamine(tromethamine).

When the compound (I) to be used for the present invention comprises a basic nitrogen-containing group, the compound may be quaternized with such agents as (C₁₋₄)alkyl halides, e.g., methyl, ethyl, iso-propyl and tert-butyl chlorides, bromides and iodides; di(C₁₋₄)alkyl sulfates, e.g., dimethyl, diethyl and diamyl sulfates; (C₁₀₋₁₈)alkyl halides, e.g., decyl, dodecyl, lauryl, myristyl and stearyl chlorides, bromides and iodides; and aryl(C₁₋₄)alkyl halides, e.g., benzyl chloride and phenethyl bromide.

N-oxides of the compound (I) can be prepared by methods known to those of ordinary skill in the art. For example, the N-oxides can be prepared by treating an unoxidized form of the compound with an oxidizing agent (e.g., trifluoroperacetic acid, permaleic acid, perbenzoic acid, peracetic acid, meta-chloroperoxybenzoic acid, or the like) in a suitable inert organic solvent (e.g., a halogenated hydrocarbon such as dichloromethane) at approximately 0° C. Alternatively, the N-oxides of the compound (I) can be prepared from the N-oxide of an appropriate starting material.

Prodrug derivatives of the compound (I) to be used for the present invention can be prepared by modifying substituents of the compounds that are then converted in vivo to a different substituent. It is noted that in many instances, the prodrugs themselves also fall within the scope of the range of the compound (I) to be used for the present invention. For example, prodrugs can be prepared by reacting a compound (I) with a carbamylating agent (e.g., 1,1-acyloxyalkylcarbonochloridate, para-nitrophenyl carbonate, or the like) or an acylating agent. Further examples of methods of making prodrugs are described in Saulnier et al. (1994), Bioorganic and Medicinal Chemistry Letters, Vol. 4, p. 1985.

Protected derivatives of the compound (I) to be used for the present invention can also be made. Examples of techniques applicable to the creation of protecting groups and their removal can be found in T. W. Greene, Protecting Groups in Organic Synthesis, 3^{rd} edition, John Wiley & Sons, Inc. 1999.

The compound (I) to be used for the present invention may also be conveniently prepared, or formed during the process of the invention, as solvates (e.g. hydrates). Hydrates of the compound (I) may be conveniently prepared by recrystallization from an aqueous/organic solvent mixture, using organic solvents such as dioxin, tetrahydrofuran and methanol.

A “pharmaceutically acceptable salt”, as used herein, is intended to encompass any compound (I) to be used for the present invention that is utilized in the form of a salt thereof, especially where the salt confers on the compound improved pharmacokinetic properties as compared to a free form of the compound or a different salt form of the compound. The pharmaceutically acceptable salt form may also initially confer desirable pharmacokinetic properties on the compound that it did not previously possess, and may even positively affect the pharmacodynamics of the compound with respect to its therapeutic activity in the body. An example of pharmacokinetic properties that may be favorably affected is the manner in which the compound is transported across cell membranes, which in turn may directly and positively affect the absorption, distribution, biotransformation and excretion of the compound. While the route of administration of the pharmaceutical composition is important, and various anatomical, physiological and pathological factors can critically affect bioavailability, the solubility of the compound is usually dependent upon the character of the particular salt form thereof, which it utilized. One of skill in the art will appreciate that an aqueous solution of the compound will provide the most rapid absorption of the compound into the body of a subject being treated, while lipid solutions and suspensions, as well as solid dosage forms, will result in less rapid absorption of the compound.

The compound (I), a salt thereof and a prodrug thereof to be used for the present invention can be produced according to a method known per se, for example, a production method described in WO2007-044779, US2007-0117816 or a method analogous thereto.

The cyclodextrin derivative to be used for the present invention may be a commercially available one or can be produced by a method known per se. The cyclodextrin derivative to be used for the present invention may be readily soluble in water, and preferably a compound wherein hydrogen(s) of a part of or all hydroxyl groups at the 2-, 3- and 6-positions of glucose of cyclic oligosaccharide consisting of 6-12 glucose units is(are) substituted by other functional group (e.g., a dihydroxyalkyl group, a saccharide residue, a hydroxyalkyl group and the like) and the like. The cyclodextrin derivative to be used for the present invention is preferably selected from a hydroxyalkyl cyclodextrin, a glucosyl cyclodextrin, a maltosyl cyclodextrin and a sulfoalkyl ether cyclodextrin, wherein the substituent(s) is (are) hydroxyalkyl group, glucosyl group, maltosyl group and sulfoalkyl group, respectively.

Said cyclodextrin derivative readily soluble in water shows solubility in water of not less than about 10 mg/ml, preferably not less than about 100 mg/ml.

Preferable examples of the cyclodextrin derivatives to be used for the present invention include a compound represented by the formula (II):

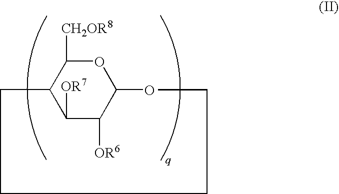

wherein q is an integer of 6-12, and R⁶, R⁷ and R⁸ are the same or different in individual repeating unit and each is a hydrogen atom, a dihydroxyalkyl group, a saccharide residue or a hydroxyalkyl group, and at least one of R⁶, R⁷ and R⁸ is a dihydroxyalkyl group, a saccharide residue or a hydroxyalkyl group.

Concrete examples thereof include ether derivatives at hydroxyl group(s) of α-CyD (q=6), β-CyD (q=7), γ-CyD (q=8), δ-CyD (q=9) and the like. Of these, an ether derivative at hydroxyl group(s) of β-CyD is preferable (in the present specification, CyD means cyclodextrin).

The dihydroxyalkyl group represented by R⁶-R⁸ is, for example, dihydroxy-C₁₋₆ alkyl group (e.g., dihydroxymethyl, 2,2-dihydroxyethyl, 2,2-dihydroxypropyl, 2,2-dihydroxypentyl, 2,2-dihydroxyhexyl and the like), preferably dihydroxy-C₁₋₄ alkyl group (e.g., dihydroxymethyl, 2,2-dihydroxyethyl, 2,2-dihydroxypropyl and the like).

The saccharide residue represented by R⁶-R⁸ is, for example, C₃₋₂₄ saccharide residue (erythrosyl, threosyl, arabinosyl, ribosyl, glucosyl, galactosyl, glycero-gluco-heptosyl, maltosyl, lactosyl, maltotriosyl, dimaltosyl and the like), preferably C₆₋₂₄ saccharide residue (e.g., glucosyl, galactosyl, glycero-gluco-heptosyl, maltosyl, lactosyl, maltotriosyl, dimaltosyl and the like), particularly preferably C₆₋₁₂ saccharide residue (e.g., glucosyl, galactosyl, glycero-gluco-heptosyl, maltosyl, lactosyl and the like).

The hydroxyalkyl group represented by R⁶-R⁸ is, for example, hydroxy-C₁₋₆ alkyl group (e.g., hydroxymethyl, 2-hydroxyethyl, 2-hydroxypropyl, 2-hydroxypentyl, 2-hydroxyhexyl and the like), preferably hydroxy-C₁₋₄ alkyl group (e.g., hydroxymethyl, 2-hydroxyethyl, 2-hydroxypropyl and the like), particularly preferably 2-hydroxypropyl group.

More preferable examples of the cyclodextrin derivative include a compound represented by the formula (II) (hereinafter to be abbreviated as cyclodextrin derivative (II)) wherein at least one of R⁶-R⁸ is a saccharide residue or a hydroxyalkyl group, and the rest is hydrogen atom.

A cyclodextrin derivative (II) wherein at least one of R⁶—R⁸ is a saccharide residue, and the rest is hydrogen atom is, for example, glucosyl-α,β,γ,δ-CyD, maltosyl-α,β,γ,δ-CyD, maltotriosyl-α,β,γ,δ-CyD, dimaltosyl-α,β,γ,δ-CyD or the like. Of these, maltosyl-α,β,γ,δ-CyD and glucosyl-α,β,γ,δ-CyD are preferable (in the present specification, α,β,γ,δ-CyD means α-CyD, β-CyD, γ-CyD or γ-CyD). Furthermore, maltosyl-β-CyD (hereinafter to be abbreviated as G2-β-CyD) and glucosyl-β-CyD are particularly preferable.

A cyclodextrin derivative wherein at least one of R⁶-R⁸ is a hydroxyalkyl group, and the rest is hydrogen atom is, for example, hydroxypropyl-α,β,γ,δ-CyD (particularly, 2-hydroxypropyl-α,β,γ,δ-CyD) or the like. Of these, hydroxypropyl-β-CyD (particularly, 2-hydroxypropyl-β-CyD) is more preferable.

The cyclodextrin derivative to be used for the present invention may be a sulfoalkyl ether cyclodextrin derivative. These sulfoalkyl ether cyclodextrin derivatives are described in U.S. Pat. No. 5,134,127, Stella et al., incorporated herein by reference, as the cyclodextrin derivatives wherein the glucopyranose units are substituted by (C₂₋₆ alkylene)-SO₃⁻ groups. These sulfoalkyl ether cyclodextrin derivatives can be used alone or in combination thereof, or as mixtures of their free form and salts thereof. Preferable salt of sulfoalkyl ether cyclodextrin is sodium salt.

In a preferred embodiment (1),

the sulfoalkyl ether cyclodextrin derivatives to be used for the present invention have structures represented by the formula (III):

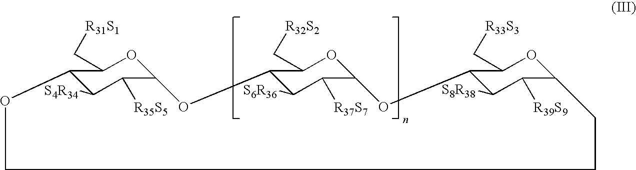

wherein n is 4, 5 or 6;
R₃₁, R₃₂, R₃₃, R₃₄, R₃₅, R₃₆, R₃₇, R₃₈ and R₃₉ are each, independently, O⁻ or a O—(C₂₋₆ alkylene)-SO₃⁻ group, wherein at least one of R₂₁ and R₂₂ is independently a O—(C₂₋₆ alkylene)-SO₃⁻ group, preferably a O—(CH₂)ₘ—SO₃⁻ group, wherein m is 2 to 6, preferably 2 to 4, (e.g. OCH₂CH₂CH₂—SO₃⁻ or OCH₂CH₂CH₂CH₂—SO₃⁻); and S₁, S₂, S₃, S₄, S₅, S₆, S₇, S₈ and S₉ are each, independently, a pharmaceutically acceptable cation which includes, for example, H⁺, alkali metals (e.g. Li⁺, Na⁺, K⁺), alkaline earth metals (e.g., Ca²⁺, Mg²⁺), ammonium ions and amines cations such as the cations C₁₋₆ alkylamines, piperidine, pyrazine, C₁₋₆ alkanolamine and C₄₋₉ cycloalkanolamine.

In another preferred embodiment (2),

R₃₁ is a O—(C₂₋₆ alkylene)-SO₃⁻ group, preferably a O—(CH₂)ₘ—SO₃⁻ group, (e.g. OCH₂CH₂CH₂—SO₃⁻ or OCH₂CH₂CH₂CH₂—SO₃⁻);

### R₃₂ to R₃₉ are O⁻;

S₁ to S₉ are as defined in embodiment (1) supra.

In another preferred embodiment (3),

R₃₁, R₃₂ and R₃₃ are each, independently, a O—(C₂₋₆ alkylene)-SO₃⁻ group, preferably a O—(CH₂)ₘ—SO₃⁻ group, (e.g. OCH₂CH₂CH₂—SO₃⁻ or OCH₂CH₂CH₂CH₂—SO₃⁻);

### R₃₄ to R₃₉ are O⁻; and

S₁ to S₉ are as defined in embodiment (1) supra.

In another preferred embodiment (4),

R₃₁ to R₃₃ are as defined in embodiments (2) or (3) supra;
at least one of R₃₄, R₃₆ and R₃₈ is a O—(C₂₋₆ alkylene)-SO₃⁻ group, preferably a O—(CH₂)ₘ—SO₃⁻ group (e.g. OCH₂CH₂CH₂—SO₃⁻ or OCH₂CH₂CH₂CH₂—SO₃⁻);

### R₃₅, R₃₇ and R₃₉ are O⁻; and

S₁ to S₉ are as defined in embodiment (1) supra.

In another preferred embodiment (5):

R₃₁, R₃₂, R₃₃, R₃₄, R₃₆ and R₃₈ are each, independently, a O—(C₂₋₆ alkylene)-SO₃⁻ group, preferably a O—(CH₂)ₘ—SO₃⁻ group (e.g., OCH₂CH₂CH₂—SO₃⁻ or OCH₂CH₂CH₂CH₂—SO₃⁻);

### R₃₅, R₃₇ and R₃₉ are O⁻; and

S₁ to S₉ are as defined in embodiment (1) supra.

The terms “alkylene” and “alkyl” in this text (e.g., in the O—(C₂₋₆ alkylene)-SO₃⁻ group or in the alkylamines) include both linear and branched, saturated and unsaturated (i.e., containing one double bond) divalent alkylene groups and monovalent alkyl groups, respectively.

The term “alkanol” in this text likewise includes both linear and branched, saturated and unsaturated alkyl components of the alkanol groups, in which the hydroxyl groups may be situated at any position on the alkyl moiety.

The term “cycloalkanol” includes unsubstituted or substituted (e.g., by methyl or ethyl)cyclic alcohols.

As the cyclodextrin derivative, a mixture of cyclodextrin derivatives having the structure set out in formula (III) (hereinafter to be also abbreviated as cyclodextrin derivative (III)) may be used for the present invention.

The cyclodextrin derivatives (III) to be used for the present invention are either substituted at least at one of the primary hydroxyl group (i.e., at least one of R₂₁ to R₂₃ is a substituent), or at both the primary hydroxyl group and at the 3-position hydroxyl group (i.e., both at least one of R₂₁ to R₂₃ and at least one of R₂₄, R₂₆ and R₂₈ are substituents). Substitution at the 2-position hydroxyl group, while theoretically possible, on the basis of the inventors' studies, does not appear to be substantial in the products of the invention.

The cyclodextrin derivative (III) to be used for the present invention is preferably sulfobutyl ether-β-cyclodextrin or a salt thereof. The sulfobutyl ether-β-cyclodextrin in the pharmaceutical composition of the present invention is preferably mono, tetra and hepta dominated substituted β-Cyclodextrin, more preferably hepta dominated substituted β-cyclodextrin. Preferable sulfobutyl ether-β-cyclodextrin derivative has an average of seven sulfobutyl ether groups per cyclodextrin molecule. Preferable salt of sulfobutyl ether-β-cyclodextrin is sodium salt, and is able to be obtained from Cydex, Inc., which is named as Captisol (registered trademark).

These cyclodextrin derivatives may be used alone or used in combination with two or more kinds thereof. The total amount of the cyclodextrin derivative(s) to be used is not particularly limited and may be determined from a wide range. In consideration of the water solubility of these substances, a cyclodextrin derivative is combined in the range of about 0.01 to about 10 mol, preferably about 0.05 to about 8 mol, further preferably about 0.1 to about 6 mol, more preferably about 0.2 to about 4 mol, relative to 1 mol of compound (I).

The content of compound (I) in the pharmaceutical composition of the present invention varies depending on the form of the preparation, but it is generally about 0.01 to about 99 wt %, preferably about 0.1 to about 50 wt %, more preferably about 0.5 to about 20 wt %, of the whole preparation.

When the pharmaceutical composition of the present invention is an injection, the content of compound (I) is generally about 0.1 to about 10 wt %, preferably about 0.1 to about 6 wt %, more preferably about 0.5 to about 5 wt %, particularly preferably about 0.5 to about 4 wt %, of the whole preparation.

The content of cyclodextrin derivative(s) in the pharmaceutical composition of the present invention varies depending on the form of the preparation, but it is generally about 0.01 to about 90 w/v %, preferably about 0.05 to about 85 w/v %, more preferably about 0.05 to about 70 w/v %, particularly preferably about 0.1 to about 50 w/v %, of the whole preparation.

When the pharmaceutical composition of the present invention is an injection, the content of cyclodextrin derivative(s) is generally about 0.01 to about 85 w/v %, preferably about 0.05 to about 70 w/v %, more preferably about 1 to about 50 w/v %, particularly preferably about 2 to about 30 w/v %, of the whole preparation.

The composition for injection of the present invention thus obtained can be freeze-dried in an aseptically treated freeze dryer and preserved in a powder state, or can be sealed in a container for injection (e.g., ampoule) and preserved.

In addition, the pharmaceutical composition of the present invention can be diluted with the aforementioned carrier for injection when in use.

As described above, the pharmaceutical composition of the present invention, particularly the composition for injection, has improved water-solubility, solubility or (and) stability of a compound (I). Therefore, it is highly safe for human and can be used to mammals (e.g., rat, mouse, guinea pig, monkey, bovine, dog, pig, cat, horse, goat, human and the like) as a pharmaceutical agent (e.g., agent for prophylaxis or therapy of various diseases), veterinary drugs and the like. The composition for injection of the present invention can be administered intravenously, intramuscularly, subcutaneously, into the organs or directly into the lesion.

When the pharmaceutical composition of the present invention is an injection, the pH is desirably adjusted to about 2 to 5, preferably about 2 to 4.5, more preferably about 2 to 4. A pharmaceutical composition of the present invention has low toxicity and can be administered safely according to a method known per se as a pharmaceutical composition, such as tablets (inclusive of sugar-coated tablets and film-coated tablets), powders, granules, capsules, (inclusive of soft capsules), liquids, injections, suppositories, sustained release agents and the like, for oral or parenteral administration (e.g., topical, rectal or intravenous administration). The pharmaceutical composition of the present invention is preferably an injection, more preferably a non-emulsified composition, a solution or a clear injection.

In the present invention, by the “non-emulsified composition” is meant a composition other than an emulsion, or a composition which is not an O/W type emulsion or a W/O type emulsion. In other words, when two solutions are mixed, phase separation, or emulsification wherein one phase is dispersed in the other phase in a fine particle state, does not occur, but a composition having a single phase, which is a uniform mixture.

In the present invention, “being clear” means a state free of cloudiness by visual oil drop or particles.

Since a compound (I) has low toxicity and kinase inhibitory effect, the kinase is optionally Aurora kinase such as Aurora-A, Aurora-B, Aurora-C, etc., in particular, the kinase is an Aurora-B kinase, the composition of the present invention, which contains the compound (I), a salt thereof or a prodrug thereof is useful as a therapeutic and/or prophylactic agent in a mammal (e.g., rat, mouse, guinea pig, monkey, bovine, dog, pig, cat, horse, goat, human and the like) against a disease state for which a kinase possesses activity that contributes to the pathology and/or symptomology of the disease state, comprising administration to a mammalian species in need thereof of a therapeutically effective amount of a composition.

The composition of the present invention, which contains a compound (I), a salt thereof or a prodrug thereof is useful as a therapeutic and/or prophylactic agent in a mammal (e.g., rat, mouse, guinea pig, monkey, bovine, dog, pig, cat, horse, goat, human and the like) against cancer, inflammation, inflammatory bowel disease, psoriasis, transplant rejection, amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss, contraceptive medication, mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment, cognitive impairment, androgenetic alopecia, dementia related diseases, Alzheimer's Disease, conditions associated with kinases, arthritis or the like, comprising administration to a mammalian species in need thereof of a therapeutically effective amount of the composition.

In one particular variation, the cancer is selected from the group consisting of squamous cell carcinoma, astrocytoma, Kaposi's sarcoma, glioblastoma, non small-cell lung cancer, bladder cancer, head and neck cancer, melanoma, ovarian cancer, prostate cancer, breast cancer, small-cell lung cancer, glioma, colorectal cancer, genitourinary cancer, gastrointestinal cancer, thyroid cancer and skin cancer.

In one particular variation, the dementia related diseases are selected from the group consisting of Frontotemporal dementia Parkinson's Type, Parkinson dementia complex of Guam, HIV dementia, diseases with associated neurofibrillar tangle pathologies, predemented states, vascular dementia, dementia with Lewy bodies, Frontotemporal dementia and dementia pugilistica and the like.

While the dose of the pharmaceutical composition of the present invention may vary depending on the kind of compound (I), age, body weight and condition, the dosage form, the mode and the period of the treatment, etc., it may, for example, be generally about 0.01 to about 1000 mg/kg, preferably about 0.01 to about 100 mg/kg, more preferably about 0.1 to about 100 mg/kg, most preferably about 0.1 to about 50 mg/kg, and particularly about 1.5 to about 30 mg/kg, as compound (I), per day in a patient having a cancer (adult weighing about 60 kg), said daily dose being given intravenously all at once or in several portions during a day. It is a matter of course that a lower daily dose may be sufficient or an excessive dose may be required since the dose may vary depending on various factors as discussed above.

In the present invention, the “effective amount” means an effective amount of compound (I) and “administration of an effective amount” means administering the pharmaceutical composition of the present invention containing an effective amount of compound (I).

As the pharmacologically acceptable carrier usable for the production of the preparation of the present invention, there are mentioned various conventional organic or inorganic carriers as a material for the preparation. Examples thereof include excipients, lubricants, binders, disintegrators, solvents, solubilizers, suspending agents, isotonic agents, buffering agents, local anesthetics, pH adjusting agents, and the like.

Where necessary, conventional additives such as antiseptics, antioxidants, coloring agents, sweeteners, absorbents, moistening agents and the like can be used appropriately in suitable amounts.

The content of other additive in the pharmaceutical composition of the present invention varies depending on the form of the preparation, but it is generally about 0.1 to about 99.9 wt %, preferably about 0.1 to about 50 wt %, more preferably about 0.1 to about 25 wt %, particularly preferably about 0.2 to about 5 wt % of the whole preparation.

As the excipient, there are mentioned, for example, lactose, sucrose, D-mannitol, starch, corn starch, crystalline cellulose, light anhydrous silicic acid and the like.

As the lubricant, there are mentioned, for example, magnesium stearate, calcium stearate, talc, colloidal silica and the like.

As the binder, there are mentioned, for example, crystalline cellulose, sucrose, D-mannitol, dextrin, hydroxypropylcellulose, hydroxypropylmethylcellulose, polyvinylpyrrolidone, starch, sucrose, gelatin, methylcellulose, carboxymethylcellulose sodium and the like.

As the disintegrator, there are mentioned, for example, starch, carboxymethylcellulose, carboxymethylcellulose calcium, sodium carboxymethyl starch, L-hydroxypropylcellulose and the like.

As the solvent, there are mentioned, for example, water for injection, alcohol, propylene glycol, Macrogol, sesame oil, corn oil, olive oil and the like.

As the solubilizer, there are mentioned, for example, polyethylene glycol, propylene glycol, D-mannitol, benzyl benzoate, ethanol, tris-aminomethane, cholesterol, triethanolamine, sodium carbonate, sodium citrate, a nonionic surfactant, an anionic surfactant, a cationic surfactant, an amphoteric surfactant and the like.

As the suspending agent, there are mentioned, for example, surfactants such as stearyl triethanolamine, sodium lauryl sulfate, lauryl aminopropionate, lecithin, benzalkonium chloride, benzethonium chloride, glyceryl monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose and the like, and the like.

As the isotonic agent, there are mentioned, for example, glucose, D-sorbitol, sodium chloride, glycerine, D-mannitol and the like.

As the buffering agent, there are mentioned, for example, buffers such as phosphate, acetate, tartrate, carbonate, citrate etc., and the like.

As the local anesthetics, there are mentioned, for example, benzyl alcohol and the like.

As the pH adjusting agent, there are mentioned, for example, hydrochloric acid, phosphoric acid, citric acid, sodium hydroxide and the like.

As the antiseptic, there are mentioned, for example, p-oxybenzoates, chlorobutanol, benzyl alcohol, phenethyl alcohol, dehydroacetic acid, sorbic acid and the like.

As the antioxidant, there are mentioned, for example, sulfite, ascorbic acid, α-tocopherol and the like.

Examples of the buffering agent include buffers such as phosphate, acetate, tartrate, carbonate, citrate and the like.

Particularly, when the pharmaceutical composition of the present invention is used as an injection, a carrier for injection to be used is exemplified by a solvent, a solubilizer, a suspending agent, an isotonic agent, a buffering agent, local anesthetics, pH adjusting agent and the like. Examples of the solvent include water for injection, physiological saline, Ringer's solution and the like. Examples of the solubilizer include polyethylene glycol, propylene glycol, D-mannitol, benzyl benzoate, tris-aminomethane, cholesterol, triethanolamine, sodium carbonate, sodium citrate and the like.

Examples of the suspending agent include stearyl triethanolamine, sodium lauryl sulfate, lauryl aminopropionate, lecithin, benzalkonium chloride, benzethonium chloride, glyceryl monostearate and the like; hydrophilic polymers such as polyvinyl alcohol, polyvinylpyrrolidone, carboxymethylcellulose sodium, methylcellulose, hydroxymethylcellulose, hydroxyethylcellulose, hydroxypropylcellulose, and the like.

Examples of the isotonic agent include glucose, D-sorbitol, sodium chloride, glycerin, D-mannitol and the like. Examples of the buffering agent include buffers such as phosphate, acetate, tartrate, carbonate, citrate, and the like.

Examples of the local anesthetics include benzyl alcohol and the like.

Examples of the pH adjusting agent include hydrochloric acid, phosphoric acid, citric acid, sodium hydroxide and the like.

The nonionic surfactant to be used as a solubilizer for the pharmaceutical composition of the present invention is, for example, a higher alcohol ethyleneoxide adduct, an alkylphenol ethyleneoxide adduct, a fatty acid ethyleneoxide adduct, a polyhydric alcohol fatty acid ester ethyleneoxide adduct, a higher alkylamine ethyleneoxide adduct, a fatty acid amide ethyleneoxide adduct, an ethyleneoxide adduct of fat and oil, a polypropylene glycol ethyleneoxide adduct, a fatty acid ester of glycerol, a fatty acid ester of pentaerythritol, a fatty acid ester of sorbitol or sorbitan, a fatty acid ester of sucrose, an alkyl ether of polyhydric alcohol, a fatty acid amide of alkanolamines, a polyoxyethylene castor oil derivative and the like.

As the anionic surfactant to be used as a solubilizer for the pharmaceutical composition of the present invention, for example, sulfuric acid esters (e.g., a higher alcohol sulfuric acid ester salt, a higher alkyl ether sulfuric acid ester salt, a sulfated oil, a sulfated fatty acid ester, a sulfated fatty acid, a sulfated olefin), sulfonic acid salts (e.g., sodium alkylbenzenesulfonate, oil soluble alkylbenzenesulfonic acid salt, α-olefinsulfonic acid salt, Igepon T type, Aerosol OT type), phosphoric acid esters (e.g., a phosphoric acid ester salt of higher alcohol ethyleneoxide adduct), a dithiophosphoric acid ester salt and the like are used.

As the cationic surfactant to be used as a solubilizer for the pharmaceutical composition of the present invention, for example, amine salt type cationic surfactants (e.g., an amine salt type cationic surfactant made from higher alkylamine, an amine salt type cationic surfactant made from lower or higher alkylamine), quaternary ammonium salt type cationic surfactants (e.g., a quaternary ammonium salt type cationic surfactant made from higher alkylamine, a quaternary ammonium salt type surfactant made from lower or higher alkylamine) and the like are used.

As the amphoteric surfactant to be used as a solubilizer for the pharmaceutical composition of the present invention, for example, an amino acid type amphoteric surfactant, a betaine type amphoteric surfactant and the like are used.

The pharmaceutical composition of the present invention preferably contains citric acid. The concentration of citric acid in the pharmaceutical composition of the present invention varies depending on the form of the preparation, but it is generally about 1 to about 500 mmol/L, preferably about 1 to about 300 mmol/L, more preferably about 1 to about 200 mmol/L, particularly preferably about 10 to about 100 mmol/L.

The present invention also provides a method of improving stability in water of a compound (I), a salt thereof or a prodrug thereof, which comprises combining the compound (I), a salt thereof or a prodrug thereof with at least one cyclodextrin derivative. In addition, there is provided a method of improving solubility in water of a compound (I), a salt thereof or a prodrug thereof, which comprises combining the compound (I), a salt thereof or a prodrug thereof with at least one cyclodextrin derivative.

In the present invention, by the “combining a compound with a cyclodextrin derivative” is meant mixing a compound and a cyclodextrin derivative, and includes forming an inclusion compound or a complex formed by electrostatic or hydrophobic interactions or hydrogen bonds, etc. between a compound and a cyclodextrin derivative.

In a preferable embodiment, the pharmaceutical composition of the present invention comprises

i) 5-(3-(ethylsulfonyl)phenyl)-3,8-dimethyl-N-(1-methylpiperidin-4-yl)-9H-pyrido[2,3-b]indole-7-carboxamide or a salt thereof,
ii) sulfobutyl ether-β-cyclodextrin or a salt thereof, and
iii) water.

In another preferable embodiment, the pharmaceutical composition of the present invention comprises

i) 5-(3-(ethylsulfonyl)phenyl)-3,8-dimethyl-N-(1-methylpiperidin-4-yl)-9H-pyrido[2,3-b]indole-7-carboxamide or a salt thereof,
ii) sulfobutyl ether-β-cyclodextrin or a salt thereof,
iii) water and
iv) citric acid,
which is in the form of an injectable composition having a pH of about 2 to about 4.

Such preferable composition is prepared by a method comprising;

i) dissolving a) 5-(3-(ethylsulfonyl)phenyl)-3,8-dimethyl-N-(1-methylpiperidin-4-yl)-9H-pyrido[2,3-b]indole-7-carboxamide or a salt thereof, b) sulfobutyl ether-β-cyclodextrin or a salt thereof and c) citric acid in water to give a solution, and
ii) adjusting pH of the solution to about 2 to about 4.

For formulation of the pharmaceutical composition of the present invention, the order of dissolution of the components such as i) 5-(3-(ethylsulfonyl)phenyl)-3,8-dimethyl-N-(1-methylpiperidin-4-yl)-9H-pyrido[2,3-b]indole-7-carboxamide or a salt thereof, ii) sulfobutyl ether-β-cyclodextrin or a salt thereof and iii) citric acid is not particularly limited, and each component may be separately dissolved or two or more kinds of components may be dissolved simultaneously. When the pharmaceutical composition of the present invention is to be formulated by separately dissolving each component, for example, sulfobutyl ether-β-cyclodextrin or a salt thereof is dissolved in water to give a solution, citric acid and the like are dissolved in the solution, 5-(3-(ethylsulfonyl)phenyl)-3,8-dimethyl-N-(1-methylpiperidin-4-yl)-9H-pyrido[2,3-b]indole-7-carboxamide or a salt thereof is further dissolved therein, and the solution is adjusted to pH about 2 to about 4. The pH can be adjusted using aqueous NaOH solution or aqueous HCl solution.

In still another preferable embodiment, the pharmaceutical composition of the present invention comprises

i) 5 to 40 mg/mL of 5-(3-(ethylsulfonyl)phenyl)-3,8-dimethyl-N-(1-methylpiperidin-4-yl)-9H-pyrido[2,3-b]indole-7-carboxamide or a salt thereof,
ii) 2 to 30 w/v % of sulfobutyl ether-β-cyclodextrin or a salt thereof,
iii) water and
iv) 10 to 100 mmol/L of citric acid,
which is in the form of an injectable composition having a pH of about 2 to about 4.

In the above-mentioned preferable embodiments, the sulfobutyl ether-β-cyclodextrin or a salt thereof is preferably sulfobutyl ether-β-cyclodextrin sodium salt.

In yet another preferable embodiment, the pharmaceutical composition of the present invention comprises

i) the compound (I), a salt thereof or a prodrug thereof, and
ii) sulfobutyl ether-β-cyclodextrin sodium salt.

In a more preferable embodiment, the pharmaceutical composition of the present invention comprises

i) 5-(3-(ethylsulfonyl)phenyl)-3,8-dimethyl-N-(1-methylpiperidin-4-yl)-9H-pyrido[2,3-b]indole-7-carboxamide or a salt thereof, and
ii) sulfobutyl ether-β-cyclodextrin or a salt thereof.

In a still more preferable embodiment, the pharmaceutical composition of the present invention comprises

i) 5-(3-(ethylsulfonyl)phenyl)-3,8-dimethyl-N-(1-methylpiperidin-4-yl)-9H-pyrido[2,3-b]indole-7-carboxamide or a salt thereof, and
ii) sulfobutyl ether-β-cyclodextrin sodium salt.

### EXAMPLES

The present invention is explained in detail in the following by referring to Reference Examples, Examples, Comparative Examples and Experimental Examples, which are not to be construed as limitative.

The proportion of the mixed solvent is a weight mixing ratio of each solvent, wherein % means % by weight unless otherwise specified.

The following Reference Examples A can be produced according to Examples of WO2007/044779 and US2007/0117816.

### Reference Examples A

Particular examples of compounds (I) involved in the pharmaceutical composition of the present invention include, but are not limited to:
5-bromo-9H-pyrido[2,3-b]indole;
5-bromo-8-methyl-9H-pyrido[2,3-b]indole;
5-bromo-3,8-dimethyl-9H-pyrido[2,3-b]indole;
5-phenyl-9H-pyrido[2,3-b]indole;
5-(3-(methylsulfonyl)phenyl)-9H-pyrido[2,3-b]indole;
5-(3-(ethylsulfonyl)phenyl)-9H-pyrido[2,3-b]indole;
N-(3-(9H-pyrido[2,3-b]indol-5-yl)phenyl)ethanesulfonamide;
5-m-tolyl-9H-pyrido[2,3-b]indole;
N-cyclopropyl-3-(9H-pyrido[2,3-b]indol-5-yl)benzenesulfonamide;
5-(3-methoxyphenyl)-9H-pyrido[2,3-b]indole;
5-(3,8-dimethyl-9H-pyrido[2,3-b]indol-5-yl)-2-methoxy-N-methylbenzenesulfonamide;
3-(3,8-dimethyl-9H-pyrido[2,3-b]indol-5-yl)-N-methylbenzenesulfonamide;
3-(3,8-dimethyl-9H-pyrido[2,3-b]indol-5-yl)-N,N-dimethylbenzenesulfonamide;
5-(3-(ethylsulfonyl)phenyl)-8-methyl-9H-pyrido[2,3-b]indole;
5-(3-(ethylsulfonyl)phenyl)-3,8-dimethyl-9H-pyrido[2,3-b]indole;
N-(3-(3,8-dimethyl-9H-pyrido[2,3-b]indol-5-yl)phenyl)propionamide;
N-cyclopropyl-3-(3,8-dimethyl-9H-pyrido[2,3-b]indol-5-yl)benzamide;
N-(4-(9H-pyrido[2,3-b]indol-5-ylthio)phenyl)acetamide;
5-(benzylthio)-9H-pyrido[2,3-b]indole;
5-(phenylthio)-9H-pyrido[2,3-b]indole;
5-(benzylthio)-8-methyl-9H-pyrido[2,3-b]indole;
5-(benzylthio)-3,8-dimethyl-9H-pyrido[2,3-b]indole;
7-benzyl-5-(3-ethanesulfonyl-phenyl)-3-methyl-7,9-dihydro-dipyrido[2,3-b;4′,3′-d]pyrrol-8-one;
8-chloro-5-(3-ethanesulfonyl-phenyl)-3-methyl-9H-dipyrido[2,3-b;4′,3′-d]pyrrole;
N′-[5-(3-ethanesulfonyl-phenyl)-3-methyl-9H-dipyrido[2,3-b;4′,3′-d]pyrrol-8-yl]-N,N-dimethyl-ropane-1,3-diamine;
N′-[5-(3-ethanesulfonyl-phenyl)-3-methyl-9H-dipyrido[2,3-b;4′,3′-d]pyrrol-8-yl]-N,N-dimethyl-ethane-1,2-diamine;
[5-(3-ethanesulfonyl-phenyl)-3-methyl-9H-dipyrido[2,3-b;4′,3′-d]pyrrol-8-yl]-(3-morpholin-4-yl-propyl)-amine;
[5-(3-ethanesulfonyl-phenyl)-3-methyl-9H-dipyrido[2,3-b;4′,3′-d]pyrrol-8-yl]-(1-methyl-piperidin-4-yl)-amine;
2-[5-(3-ethanesulfonyl-phenyl)-3-methyl-9H-dipyrido[2,3-b;4′,3′-d]pyrrol-8-ylamino]-ethanol;
[5-(3-ethanesulfonyl-phenyl)-3-methyl-9H-dipyrido[2,3-b;4′,3′-d]pyrrol-8-yl]-(1-methyl-piperidin-4-ylmethyl)-amine;
5-(3-ethanesulfonyl-phenyl)-3,8-dimethyl-9H-dipyrido[2,3-b;4′,3′-d]pyrrole;
5-(3-ethanesulfonyl-phenyl)-8-ethyl-3-methyl-9H-dipyrido[2,3-b;4′,3′-d]pyrrole;
5-(3-ethanesulfonyl-phenyl)-3-methyl-9H-dipyrido[2,3-b;4′,3′-d]pyrrole-8-carbonitrile;
5-(3-ethanesulfonyl-phenyl)-3-methyl-9H-dipyrido[2,3-b;4′,3′-d]pyrrole-8-carboxylic acid amide;
5-(3-ethanesulfonyl-phenyl)-8-ethoxy-3-methyl-9H-dipyrido[2,3-b;4′,3′-d]pyrrole;
{3-[5-(3-ethanesulfonyl-phenyl)-3-methyl-9H-dipyrido[2,3-b;4′,3′-d]pyrrol-8-yloxy]-propyl}-dimethyl-amine;
2-[5-(3-ethanesulfonyl-phenyl)-3-methyl-9H-dipyrido[2,3-b;4′,3′-d]pyrrol-8-yloxy]-ethanol;
5-(3-ethanesulfonyl-phenyl)-3-methyl-8-(1-methyl-piperidin-4-ylmethoxy)-9H-dipyrido[2,3-b;4′,3′-d]pyrrole;
3-[5-(3-ethanesulfonyl-phenyl)-3-methyl-9H-dipyrido[2,3-b;4′,3′-d]pyrrol-8-yloxy]-propan-1-ol;
(R)-2-[5-(3-ethanesulfonyl-phenyl)-3-methyl-9H-dipyrido[2,3-b;4′,3′-d]pyrrol-8-yloxymethyl]-propane-1,3-diol;
(S)-2-[5-(3-ethanesulfonyl-phenyl)-3-methyl-9H-dipyrido[2,3-b;4′,3′-d]pyrrol-8-yloxymethyl]-propane-1,3-diol;
1-[5-(3-ethanesulfonyl-phenyl)-3-methyl-9H-dipyrido[2,3-b;4′,3′-d]pyrrol-8-yloxy]-2-methyl-propan-2-ol;
5-(3-ethanesulfonyl-phenyl)-3-methyl-8-phenoxy-9H-dipyrido[2,3-b;4′,3′-d]pyrrole;
5-(3-ethanesulfonyl-phenyl)-3-methyl-8-(thiazol-5-ylmethoxy)-9H-dipyrido[2,3-b;4′,3′-d]pyrrole;
5-(3-ethanesulfonyl-phenyl)-8-(1-ethyl-piperidin-4-ylmethoxy)-3-methyl-9H-dipyrido[2,3-b;4′,3′-d]pyrrole;
(S)-1-[5-(3-ethanesulfonyl-phenyl)-3-methyl-9H-dipyrido[2,3-b;4′,3′-d]pyrrol-8-yloxy]-propan-2-ol;
(R)-1-[5-(3-ethanesulfonyl-phenyl)-3-methyl-9H-dipyrido[2,3-b;4′,3′-d]pyrrol-8-yloxy]-propan-2-ol;
L-valine-2-[5-(3-ethanesulfonyl-phenyl)-3-methyl-9H-dipyrido[2,3-b;4′,3′-d]pyrrol-8-yloxy]-ethyl ester;
L-alanine-(R)-2-[5-(3-ethanesulfonyl-phenyl)-3-methyl-9H-dipyrido[2,3-b;4′,3′-d]pyrrol-8-yloxy]-1-methyl-ethyl ester;
3-(3-bromo-5-chloro-pyridin-2-ylamino)-5-chloro-1-(4-methoxy-benzyl)-1H-pyrazin-2-one;
3,8-dichloro-5-(3-ethanesulfonyl-phenyl)-9H-dipyrido[2,3-b;4′,3′-d]pyrrole;
3-chloro-5-(3-ethanesulfonyl-phenyl)-8-(1-methyl-piperidin-4-ylmethoxy)-9H-dipyrido[2,3-b;4′,3′-d]pyrrole;
(R)-1-[3-chloro-5-(3-ethanesulfonyl-phenyl)-9H-dipyrido[2,3-b;4′,3′-d]pyrrol-8-yloxy]-propan-2-ol;
2-[5-(3-ethanesulfonyl-phenyl)-3-methyl-9H-dipyrido[2,3-b;4′,3′-d]pyrrol-8-yl]methyl amine;
2-[5-(3-ethanesulfonyl-phenyl)-3-methyl-9H-dipyrido[2,3-b;4′,3′-d]pyrrol-8-yl]methanethiol;
2-[5-(3-ethanesulfonyl-phenyl)-3-methyl-9H-dipyrido[2,3-b;4′,3′-d]pyrrol-8-yl]ethanethiol;
8-chloro-5-[3-(cyclopropylcarboxamide)phenyl]-3-methyl-9H-dipyrido[2,3-b;4′,3′-d]pyrrole;
2-[5-(3-cyclopropylcarbonylamino-phenyl)-3-methyl-9H-dipyrido[2,3-b;4′,3′-d]pyrrol-8-yl]ethanethiol;
9-(3-Ethanesulfonyl-phenyl)-5H-pyrazino[2,3-b]indole;
5-(3-(ethylsulfonyl)phenyl)-3,8-dimethyl-7-(trifluoromethyl)-9H-pyrido[2,3-b]indole acetate;
5-(3-(ethylsulfonyl)phenyl)-3,8-dimethyl-9H-pyrido[2,3-b]indole-7-carboxylic acid;
N-(2-(dimethylamino)ethyl)-5-(3-(ethylsulfonyl)phenyl)-3,8-dimethyl-9H-pyrido[2,3-b]indole-7-carboxamide;
N-(2-(methylamino)ethyl)-5-(3-(ethylsulfonyl)phenyl)-3,8-dimethyl-9H-pyrido[2,3-b]indole-7-carboxamide;
N-(2-(methoxy)ethyl)-5-(3-(ethylsulfonyl)phenyl)-3,8-dimethyl-9H-pyrido[2,3-b]indole-7-carboxamide;
N-(2-(dimethylamino)ethyl)-N-methyl-5-(3-(ethylsulfonyl)phenyl)-3,8-dimethyl-9H-pyrido[2,3-b]indole-7-carboxamide;
N,N-dimethyl-5-(3-(ethylsulfonyl)phenyl)-3,8-dimethyl-9H-pyrido[2,3-b]indole-7-methylcarboxamide;
5-(3-(ethylsulfonyl)phenyl)-3,8-dimethyl-9H-pyrido[2,3-b]indole-7-yl)(4-methylpiperazin-1-yl)methanone;
5-(3-(ethylsulfonyl)phenyl)-3,8-dimethyl-N-(2-piperazin-1-yl)ethyl)-9H-pyrido[2,3-b]indole-7-carboxamide;
5-(3-(ethylsulfonyl)phenyl)-3,8-dimethyl-N-(3-(4-methylpiperazin-1-yl)propyl)-9H-pyrido[2,3-b]indole-7-carboxamide;
5-(3-(ethylsulfonyl)phenyl)-3,8-dimethyl-9H-pyrido[2,3-b]indole-7-yl)(morpholino)methanone;
azetidin-1-yl(5-(3-(ethylsulfonyl)phenyl)-3,8-dimethyl-9H-pyrido[2,3-b]indol-7-yl)methanone;
(5-(3-(ethylsulfonyl)phenyl)-3,8-dimethyl-9H-pyrido[2,3-b]indol-7-yl)(thaiazolidin-3-yl)methanone;
(R)-5-(3-(ethylsulfonyl)phenyl)-N-(2-hydroxypropyl)-3,8-dimethyl-9H-pyrido[2,3-b]indole-7-carboxamide;
(S)-5-(3-(ethylsulfonyl)phenyl)-N-(2-hydroxypropyl)-3,8-dimethyl-9H-pyrido[2,3-b]indole-7-carboxamide;
5-(3-(ethylsulfonyl)phenyl)-N-(2-hydroxyethyl)-3,8-dimethyl-9H-pyrido[2,3-b]indole-7-carboxamide;
N-(2,3-dihydroxypropyl)-5-(3-(ethylsulfonyl)phenyl)-3,8-dimethyl-9H-pyrido[2,3-b]indole-7-carboxamide;
5-(3-(ethylsulfonyl)phenyl)-N-(2-hydroxy-2-methylpropyl)-3,8-dimethyl-9H-pyrido[2,3-b]indole-7-carboxamide;
5-(3-(ethylsulfonyl)phenyl)-N-(1-isopropylpiperidin-4-yl)-3,8-dimethyl-9H-pyrido[2,3-b]indole-7-carboxamide;
N-(1-ethylpiperidin-4-yl)-5-(3-(ethylsulfonyl)phenyl)-3,8-dimethyl-9H-pyrido[2,3-b]indole-7-carboxamide;
5-(3-(ethylsulfonyl)phenyl)-3,8-dimethyl-N-thiazol-2-yl)-9H-pyrido[2,3-b]indole-7-carboxamide;
5-(3-(ethylsulfonyl)phenyl)-3,8-dimethyl-N-(2-(2,2,2-trifluoroethoxy)ethyl-9H-pyrido[2,3-b]indole-7-carboxamide;
5-(3-(ethylsulfonyl)phenyl)-3,8-dimethyl-N-(piperidin-3-yl)-9H-pyrido[2,3-b]indole-7-carboxamide;
5-(3-(ethylsulfonyl)phenyl)-3,8-dimethyl-N-(piperidin-4-yl)-9H-pyrido[2,3-b]indole-7-carboxamide;
5-(3-(ethylsulfonyl)phenyl)-3,8-dimethyl-N-(piperidin-3-yl)-9H-pyrido[2,3-b]indole-7-carboxamide;
5-(3-(ethylsulfonyl)phenyl)-N-(2-(2-hydroxyethoxy)ethyl)-3,8-dimethyl-9H-pyrido[2,3-b]indole-7-carboxamide;
5-(3-(cyclopropanecarboxamido)phenyl)-N-(2-(dimethylamino)ethyl)-3,8-dimethyl-9H-pyrido[2,3-b]indole-7-carboxamide;
5-(3-(ethylsulfonyl)phenyl)-3,8-dimethyl-N-(1-methylpiperidin-4-yl)-9H-pyrido[2,3-b]indole-7-carboxamide;
5-(3-(ethylsulfonyl)phenyl)-3,8-dimethyl-N-((1-methylpiperidin-4-yl)methyl)-9H-pyrido[2,3-b]indole-7-carboxamide;
N-(3-(dimethylamino)propyl)-5-(3-(ethylsulfonyl)phenyl)-3,8-dimethyl-9H-pyrido[2,3-b]indole-7-carboxamide;
5-(3-(ethylsulfonyl)phenyl)-3,8-dimethyl-N-(2-(pyrrolidin-1-yl)ethyl)-9H-pyrido[2,3-b]indole-7-carboxamide;
(S)-5-(3-(ethylsulfonyl)phenyl)-3,8-dimethyl-N-(1-methylpiperidin-3-yl)-9H-pyrido[2,3-b]indole-7-carboxamide;
(R)-5-(3-(ethylsulfonyl)phenyl)-3,8-dimethyl-N-(1-methylpiperidin-3-yl)-9H-pyrido[2,3-b]indole-7-carboxamide;
5-chloro-3,8-dimethyl-N-(1-methylpiperidin-4-yl)-9H-pyrido[2,3-b]indole-7-carboxamide;
5-(3-(cyclopropanecarboxamido)phenyl)-3,8-dimethyl-N-(1-methyl-piperidin-4-yl)-9H-pyrido[2,3-b]indole-7-carboxamide;
5-chloro-N-(2-(dimethylamino)ethyl)-3,8-dimethyl-9H-pyrido[2,3-b]indole-7-carboxamide;
5-(3-(cyclopropylcarbamoyl)phenyl)-N-(2-(dimethylamino)ethyl)-3,8-dimethyl-9H-pyrido[2,3-b]indole-7-carboxamide;
5-amino-3-methyl-9H-pyrido[2,3-b]indole-7-carbonitrile;
5-iodo-3-methyl-9H-pyrido[2,3-b]indole-7-carbonitrile;
5-(3-ethanesulfonyl-phenyl)-3-methyl-9H-pyrido[2,3-b]indole-7-carbonitrile;
5-(3-ethanesulfonyl-phenyl)-3-methyl-9H-pyrido[2,3-b]indole-7-carboxylic acid amide;
5-amino-3-methyl-9H-pyrido[2,3-b]indole-7-carboxylic acid methyl ester;
5-iodo-3-methyl-9H-pyrido[2,3-b]indole-7-carboxylic acid methyl ester;
5-(3-ethanesulfonyl-phenyl)-3-methyl-9H-pyrido[2,3-b]indole-7-carboxylic acid methyl ester;
[5-(3-ethanesulfonyl-phenyl)-3-methyl-9H-pyrido[2,3-b]indol-7-yl]-methanol;
[5-(3-ethanesulfonyl-phenyl)-3-methyl-9H-pyrido[2,3-b]indol-7-ylmethyl]-dimethyl-amine;
5-(3-ethanesulfonyl-phenyl)-3-methyl-7-morpholin-4-ylmethyl-9H-pyrido[2,3-b]indole;
5-(3-ethanesulfonyl-phenyl)-3-methyl-7-(4-methyl-piperazin-1-ylmethyl)-9H-pyrido[2,3-b]indole;
5-(3-ethanesulfonyl-phenyl)-3-methyl-7-pyrrolidin-1-ylmethyl-9H-pyrido[2,3-b]indole;
[5-(3-ethanesulfonyl-phenyl)-3-methyl-9H-pyrido[2,3-b]indol-7-ylmethyl]-ethyl-amine;
5-(3-ethanesulfonyl-phenyl)-3-methyl-9H-pyrido[2,3-b]indole-7-carboxylic acid;
[5-(3-ethanesulfonyl-phenyl)-3-methyl-9H-pyrido[2,3-b]indol-7-yl]-(4-methyl-piperazin-1-yl)-methanone;
5-(3-ethanesulfonyl-phenyl)-3-methyl-9H-pyrido[2,3-b]indole-7-carboxylic acid (2-dimethylamino-ethyl)-amide;
5-(3-ethanesulfonyl-phenyl)-3-methyl-9H-pyrido[2,3-b]indole-7-carboxylic acid (3-dimethylamino-propyl)-amide;
5-(3-ethanesulfonyl-phenyl)-3-methyl-7-(2H-tetrazol-5-yl)-9H-pyrido[2,3-b]indole;
(3-dimethylamino-pyrrolidin-1-yl)-[5-(3-ethanesulfonyl-phenyl)-3-methyl-9H-pyrido[2,3-b]indol-7-yl]-methanone;
N-ethyl-5-(3-(ethylsulfonyl)phenyl)-3-methyl-9H-pyrido[2,3-b]indole-7-carboxamide;
6-bromo-5-(3-ethanesulfonyl-phenyl)-3-methyl-9H-pyrido[2,3-b]indole-7-carboxylic acid methyl ester;
8-bromo-5-(3-ethanesulfonyl-phenyl)-3-methyl-9H-pyrido[2,3-b]indole-7-carboxylic acid methyl ester;
6-chloro-5-(3-ethanesulfonyl-phenyl)-3-methyl-9H-pyrido[2,3-b]indole-7-carboxylic acid methyl ester;
8-chloro-5-(3-ethanesulfonyl-phenyl)-3-methyl-9H-pyrido[2,3-b]indole-7-carboxylic acid methyl ester;
5-(benzylthio)-3-methyl-9H-pyrido[2,3-b]indole-7-carboxylic acid;
5-(benzylthio)-N-(2-(dimethylamino)ethyl)-3-methyl-9H-pyrido[2,3-b]indole-7-carboxamide;
5-(3-(N-ethylsulfamoyl)phenyl)-8-methoxy-3-methyl-N-(1-methylpiperidin-4-yl)-9H-pyrido[2,3-b]indole-7-carboxamide;
5-(3-(cyclopropylsulfonyl)phenyl)-3,8-dimethyl-N-(1-methylpiperidin-4-yl)-9H-pyrido[2,3-b]indole-7-carboxamide;
5-choloro-8-methoxy-9H-pyrido[2,3-b]indole;
5-(3-(ethylsulfonyl)phenyl-8-methoxy-3-methyl-9H-pyrido[2,3-b]indole;
5-(3-(ethylsulfonyl)phenyl)-3-methyl-9H-pyrido[2,3-b]indol-8-ol;
8-methoxy-3-methyl-5-(3-(pyrrolidin-1-ylsulfonyl)phenyl)-9H-pyrido[2,3-b]indole;
(R)-8-methoxy-3-methyl-5-(3-(pyrrolidin-3-ylsulfonyl)phenyl)-9H-pyrido[2,3-b]indole;
N-cyclopropyl-4-(8-methoxy-3-methyl-9H-pyrido[2,3-b]indol-5-yl)picolinamide;
N-(3-(8-methoxy-3-methyl-9H-pyrido[2,3-b]indol-5-yl)phenyl)acetamide;
N-(3-(8-methoxy-3-methyl-9H-pyrido[2,3-b]indol-5-yl)phenyl)cyclopropanecarboxamide;
N-cyclopropyl-3-(8-methoxy-3-methyl-9H-pyrido[2,3-b]indol-5-yl)benzamide;
N,N-diethyl-3-(8-methoxy-3-methyl-9H-pyrido[2,3-b]indol-5-yl)benzamide;
5-(benzo[d][1,3]dioxol-5-yl)-8-methoxy-3-methyl-9H-pyrido[2,3-b]indole;
6-(8-methoxy-3-methyl-9H-pyrido[2,3-b]indol-5-yl)-4H-chromen-4-one;
N-(2-hydroxyethyl)-3-(8-methoxy-3-methyl-9H-pyrido[2,3-b]indol-5-yl)benzamide;
(3-(8-methoxy-3-methyl-9H-pyrido[2,3-b]indol-5-yl)phenyl)(pyrrolidin-1-yl)methanone;
N-ethyl-3-(8-methoxy-3-methyl-9H-pyrido[2,3-b]indol-5-yl)benzenesulfonamide;
8-ethoxy-5-(3-(ethylsulfonyl)phenyl)-3-methyl-9H-pyrido[2,3-b]indole;
8-(difluoromethoxy)-5-(3-(ethylsulfonyl)phenyl)-3-methyl-9H-pyrido[2,3-b]indole;
5-(3-(ethylsulfonyl)phenyl)-3-methyl-8-(2,2,2-trifluoroethoxy)-9H-pyrido[2,3-b]indole;
5-(3-(ethylsulfonyl)phenyl)-3-methyl-8-((1-methylpiperidin-4-yl)methoxy)-9H-pyrido[2,3-b]indole;
N-cyclopropyl-3-(3-methyl-8-((1-methylpiperidin-4-yl)methoxy)-9H-pyrido[2,3-b]indol-5-yl)benzamide;
5-(3-(cyclopropylsulfonyl)phenyl)-3-methyl-8-((1-methylpiperidin-4-yl)methoxy)-9H-pyrido[2,3-b]indole;
N-methyl-3-(3-methyl-8-((1-methylpiperidin-4-yl)methoxy)-9H-pyrido[2,3-b]indole-5-yl)benzenesulfonamide;
N,N-dimethyl-3-(3-methyl-8-((1-methylpiperidin-4-yl)methoxy)-9H-pyrido[2,3-b]indole-5-yl)benzenesulfonamide;
N-(3-(3-methyl-8-((1-methylpiperidin-4-yl)methoxy)-9H-pyrido[2,3-b]indol-5-yl)phenyl)cyclopropanecarboxamide;
5-(3-(ethylthio)phenyl)-3-methyl-8-((1-methylpiperidin-4-yl)methoxy)-9H-pyrido[2,3-b]indole;
5-(3-ethoxyphenyl)-3-methyl-8-((1-methylpiperidin-4-yl)methoxy)-9H-pyrido[2,3-b]indole;
5-(3-(ethylsulfonyl)phenyl)-3-methyl-8-(piperidin-4-ylmethoxy)-9H-pyrido[2,3-b]indole;
(S)-5-(3-(ethylsulfonyl)phenyl)-3-methyl-8-((1-methylpyrrolidin-3-yl)methoxy)-9H-pyrido[2,3-b]indole;
(R)-5-(3-(ethylsulfonyl)phenyl)-3-methyl-8-((1-methylpyrrolidin-3-yl)methoxy)-9H-pyrido[2,3-b]indole;
(S)-5-(3-(ethylsulfonyl)phenyl)-3-methyl-8-((1-methylpyrrolidin-2-yl)methoxy)-9H-pyrido[2,3-b]indole;
(S)-5-(3-(ethylsulfonyl)phenyl)-3-methyl-8-(pyrrolidin-3-ylmethoxy)-9H-pyrido[2,3-b]indole;
(R)-5-(3-(ethylsulfonyl)phenyl)-3-methyl-8-(pyrrolidin-3-ylmethoxy)-9H-pyrido[2,3-b]indole;
3-(5-chloro-3-methyl-9H-pyrido[2,3-b]indol-8-yloxy)-N,N-dimethylpropan-1-amine;
N-(3-(8-(3-(dimethylamino)propoxy)-3-methyl-9H-pyrido[2,3-b]indol-5-yl)phenyl)cyclopropanecarboxamide;
N-cyclopropyl-3-(8-(3-(dimethylamino)propoxy)-3-methyl-9H-pyrido[2,3-b]indol-5-yl)benzamide;
3-(8-(3-(dimethylamino)propoxy)-3-methyl-9H-pyrido[2,3-b]indol-5-yl)-N-methylbenzenesulfonamide;
3-(8-(3-(dimethylamino)propoxy)-3-methyl-9H-pyrido[2,3-b]indol-5-yl)-N—N-dimethylbenzenesulfonamide;
3-chloro-5-(3-(ethylsulfonyl)phenyl)-8-((1-methylpiperidin-4-yl)methoxy)-9H-pyrido[2,3-b]indole;
3-chloro-5-(3-(ethylsulfonyl)phenyl)-8-(piperidin-4-ylmethoxy)-9H-pyrido[2,3-b]indole;
5-(3-(ethylsulfonyl)phenyl)-8-((1-methylpiperidin-4-yl)methoxy)-3-(trifluoromethyl)-9H-pyrido[2,3-b]indole;
5-(3-(ethylsulfonyl)phenyl)-8-((1-methylpiperidin-4-yl)methoxy)-9H-pyrido[2,3-b]indole-3-carbonitrile;
2-(5-(3-(ethylsulfonyl)phenyl)-7-fluoro-3-methyl-9H-pyrido[2,3-b]indol-8-yloxy)-N,N-dimethylethanamine;
3-(3-chloro-5-(3-(ethylsulfonyl)phenyl)-7-fluoro-9H-pyrido[2,3-b]indol-8-yloxy)-N,N-dimethylpropan-1-amine;
3-(5-(3-(ethylsulfonyl)phenyl)-3-methyl-9H-pyrido[2,3-b]indol-8-yloxy)-N,N-dimethylpropan-1-amine;
2-(5-(3-(ethylsulfonyl)phenyl)-3-methyl-9H-pyrido[2,3-b]indol-8-yloxy)-N,N-dimethylethanamine;
5-(3-(ethylsulfonyl)phenyl)-8-(2-methoxyethoxy)-3-methyl-9H-pyrido[2,3-b]indole;
2-(5-(3-(ethylsulfonyl)phenyl)-3-methyl-9H-pyrido[2,3-b]indol-8-yloxy)acetonitrile;
3-(5-(3-(ethylsulfonyl)phenyl)-3-methyl-9H-pyrido[2,3-b]indol-8-yloxy)propanenitrile;
(R)-8-(1-tert-butyldiphenylsilyloxy)propan-2-yloxy)-(5-(3-(ethylsulfonyl)phenyl)-3-methyl-9H-pyrido[2,3-b]indole;
(R)-2-(5-(3-(ethylsulfonyl)phenyl)-3-methyl-9H-pyrido[2,3-b]indol-8-yloxy)propan-1-ol;
(S)-2-(5-(3-(ethylsulfonyl)phenyl)-3-methyl-9H-pyrido[2,3-b]indol-8-yloxy)propan-1-ol;
1-(5-(3-(ethylsulfonyl)phenyl)-3-methyl-9H-pyrido[2,3-b]indol-8-yloxy)propan-2-ol;
(S)-4-(5-(3-(ethylsulfonyl)phenyl)-3-methyl-9H-pyrido[2,3-b]indol-8-yloxy)-2-methylpentan-2-ol;
2-(5-(3-(ethylsulfonyl)phenyl)-3-methyl-9H-pyrido[2,3-b]indol-8-yloxy)ethanol;
3-(5-(3-(ethylsulfonyl)phenyl)-3-methyl-9H-pyrido[2,3-b]indol-8-yloxy)propan-1-ol;
3-(3-chloro-5-(3-(ethylsulfonyl)phenyl)-8-methoxy-9H-pyrido[2,3-b]indol-8-ol;
(3-chloro-5-(3-(ethylsulfonyl)phenyl)-9H-pyrido[2,3-b]indol-8-ol;
3-(3-chloro-5-(3-(ethylsulfonyl)phenyl)-9H-pyrido[2,3-b]indol-8-yloxy)-N,N-dimethylpropan-1-amine;
2-(3-chloro-5-(3-(ethylsulfonyl)phenyl)-9H-pyrido[2,3-b]indol-8-yloxy)-N,N-diethylethanamine;
2-(3-chloro-5-(3-(ethylsulfonyl)phenyl)-9H-pyrido[2,3-b]indol-8-yloxy)-N,N-dimethylethanamine;
3-chloro-5-(3-(ethylsulfonyl)phenyl)-8-(2-(pyrrolidin-1-yl)ethoxy)-9H-pyrido[2,3-b]indole;
3-chloro-5-(3-(ethylsulfonyl)phenyl)-8-(2-(4-methylpiperazin-1-yl)ethoxy)-9H-pyrido[2,3-b]indole;
2-(3-chloro-5-(3-(ethylsulfonyl)phenyl)-9H-pyrido[2,3-b]indol-8-yloxy)ethanol;
3-(3-chloro-5-(3-(ethylsulfonyl)phenyl)-9H-pyrido[2,3-b]indol-8-yloxy)propan-1-ol;
(S)-2-(5-(3-(ethylsulfonyl)phenyl)-3-methyl-9H-pyrido[2,3-b]indol-8-yloxy)ethyl 2-aminopropanoate;
(S)-3-(5-(3-(ethylsulfonyl)phenyl)-3-methyl-9H-pyrido[2,3-b]indol-8-yloxy)propyl 2-aminopropanoate;
(S)-3-(3-chloro-5-(3-(ethylsulfonyl)phenyl)-9H-pyrido[2,3-b]indol-8-yloxy)propyl 2-aminopropanoate;
(R)-8-((2,2-dimethyl-1,3-dioxolan-4-yl)methoxy-5-(3-ethylsulfonyl)phenyl)-3-methyl-9H-pyrido[2,3-b]indole;
(S)-3-(5-(3-(ethylsulfonyl)phenyl)-3-methyl-9H-pyrido[2,3-b]indol-8-yloxy)propane-1,2-diol;
(R)-3-(5-(3-(ethylsulfonyl)phenyl)-3-methyl-9H-pyrido[2,3-b]indol-8-yloxy)propane-1,2-diol;
(R)-1-(dimethylamino)-3-(5-(3-(ethylsulfonyl)phenyl)-3-methyl-9H-pyrido[2,3-b]indol-8-yloxy)propan-2-ol;
(R)-1-(5-(3-(ethylsulfonyl)phenyl)-3-methyl-9H-pyrido[2,3-b]indol-8-yloxy)propan-2-ol;
(S)-1-(5-(3-(ethylsulfonyl)phenyl)-3-methyl-9H-pyrido[2,3-b]indol-8-yloxy)propan-2-ol;
5-bromo-8-methoxy-3-methyl-9H-pyrido[2,3-b]indol-7-amine;
5-(3-(ethylsulfonyl)phenyl)-8-methoxy-3-methyl-9H-pyrido[2,3-b]indol-7-amine;
N-(3-(7-amino-8-methoxy-3-methyl-9H-pyrido[2,3-b]indol-5-yl)phenyl)-cyclopropanecarboxamide;
3-(dimethylamino)-N-(5-(3-(ethylsulfonyl)phenyl)-8-methoxy-3-methyl-9H-pyrido[2,3-b]indol-7-yl)propanamide;
N-(5-(3-(ethylsulfonyl)phenyl)-8-methoxy-3-methyl-9H-pyrido[2,3-b]indol-7-yl)-cyclopropanecarboxamide;
1-acetyl-N-(5-(3-(ethylsulfonyl)phenyl)-8-methoxy-3-methyl-9H-pyrido[2,3-b]indol-7-yl)piperidine-4-carboxamide;
3-(7-amino-8-methoxy-3-methyl-9H-pyrido[2,3-b]indol-5-yl)-N-cyclopropylbenzamide;
3-(7-(cyclopropanecarboxamido)-8-methoxy-3-methyl-9H-pyrido[2,3-b]indol-5-yl)-N-cyclopropylbenzamide;
7-chloro-5-(3-(ethylsulfonyl)phenyl)-8-methoxy-3-methyl-9H-pyrido[2,3-b]indole;
7-chloro-5-(3-(ethylsulfonyl)phenyl)-3-methyl-9H-pyrido[2,3-b]indol-8-ol;
3-(7-chloro-5-(3-(ethylsulfonyl)phenyl)-3-methyl-9H-pyrido[2,3-b]indol-8-yloxy)propan-1-ol;
N-(5-(3-(ethylsulfonyl)phenyl)-8-methoxy-3-methyl-9H-pyrido[2,3-b]indol-7-yl)-N-methylcyclopropanecarboxamide;
3-(dimethylamino)-N-(5-(3-(ethylsulfonyl)phenyl)-8-methoxy-3-methyl-9H-pyrido[2,3-b]indol-7-yl)-N-methylpropanamide;
5-(3-(cyclopropylcarbamoyl)phenyl)-3,8-dimethyl-N-(1-methylpiperidin-4-yl)-9H-pyrido[2,3-b]indole-7-carboxamide;
4-(2-(3-chloro-5-(3-(ethylsulfonyl)phenyl)-9H-pyrido[2,3-b]indol-8-yloxy)ethyl)morpholine;
3-(3-chloro-5-(3-(ethylsulfonyl)phenyl)-9H-pyrido[2,3-b]indol-8-yloxy)propanenitrile;
3-chloro-5-(3-(ethylsulfonyl)phenyl)-8-(1-methylpiperidin-4-yloxy)-9H-pyrido[2,3-b]indole;
3-(5-(3-(ethylsulfonyl)phenyl)-3-(trifluoromethyl)-9H-pyrido[2,3-b]indol-8-yloxy)-N,N-dimethylpropan-1-amine;
(3-(8-methoxy-3-methyl-9H-pyrido[2,3-b]indol-5-yl)phenyl)(morpholino)methanone;
N-methoxy-3-(8-methoxy-3-methyl-9H-pyrido[2,3-b]indol-5-yl)benzamide;
5-(3-Ethanesulfonyl-phenyl)-8-(cyclopropylmethoxy)-3-methyl-9H-dipyrido[2,3-b;4′,3′-d]pyrrole;
N-(2-(diethylamino)ethyl)-5-(3-(ethylsulfonyl)phenyl)-3,8-dimethyl-9H-pyrido[2,3-b]indole-7-carboxamide; and
5-(3-(ethylsulfonyl)phenyl)-3,8-dimethyl-N-(3-morpholinopropyl)-9H-pyrido[2,3-b]indole-7-carboxamide.

Particular examples of compounds (I) involved in the pharmaceutical composition of the present invention also include, but are not limited to:
5-(3-(ethylsulfonyl)phenyl)-3,8-dimethyl-N-(1-methylpiperidin-4-yl)-9H-pyrido[2,3-b]indole-7-carboxamide HCl salt;
5-(3-(ethylsulfonyl)phenyl)-3-methyl-8-((1-methylpiperidin-4-yl)methoxy)-9H-pyrido[2,3-b]indole HCl salt;
3-chloro-5-(3-(ethylsulfonyl)phenyl)-8-((1-methylpiperidin-4-yl)methoxy)-9H-pyrido[2,3-b]indole HCl salt;
3-(5-(3-(ethylsulfonyl)phenyl)-3-methyl-9H-pyrido[2,3-b]indol-8-yloxy)-N,N-dimethylpropan-1-amine HCl salt;
3-(3-chloro-5-(3-(ethylsulfonyl)phenyl)-9H-pyrido[2,3-b]indol-8-yloxy)-N,N-dimethylpropan-1-amine HCl salt; and
N-cyclopropyl-3-(3-methyl-8-((1-methylpiperidin-4-yl)methoxy)-9H-pyrido[2,3-b]indol-5-yl)benzamide HCl salt.

### Comparative Example 1

### Solubilizing Effect of Citric Acid Buffer Solutions at Various pH Values on 5-(3-(ethylsulfonyl)phenyl)-3,8-dimethyl-N-(1-methylpiperidin-4-yl)-9H-pyrido[2,3-b]indole-7-carboxamide (Also Sometimes Referred to as Compound A1) Hydrochloride

Each of the solutions (0.1 mL) shown in Table 1 was added to compound A1 hydrochloride (about 2 mg), and solubility was confirmed with eye observation. When insoluble materials were recognized, the solution (0.1 mL) was further added, and solubility was confirmed by eye observation in the same manner. When insoluble materials were still recognized, the solution was thereafter added in the amounts of 0.05 mL, 0.15 mL, 0.4 mL, 0.4 mL, 0.4 mL and 0.4 mL, respectively in this order, and solubility was confirmed by eye observation. The concentration of the compound A1 hydrochloride determined from the total amount of the solution added up to the time point at which insoluble materials were not recognized, was taken as the solubility.

Compared to the solubility in the water for injection, a solubilizing effect in a 100 mM citric acid buffer solution at pH 2 was recognized, but a solubilizing effect exhibiting a solubility of 8 mg/mL or more was not obtained. In addition, the symbol “<” in the table indicates that insoluble materials were recognized at the time point where the solution was added up to a total amount of 2 mL.

TABLE 1

| | Weighed value | | |
|---|---|---|---|
| | of compound | Total amount | |
| | A1 | of added | |
| Solution | hydrochloride | solution | Solubility |
| Water for | 2.4 mg | 2 mL | <1.2 mg/mL |
| injection | | | |
| 100 mM Citric | 2.5 mg | 1.2 mL | About 2.1 |
| acid buffer | | | mg/mL |
| solution pH 2 | | | |
| 100 mM Citric | 1.9 mg | 2 mL | About 1.0 |
| acid buffer | | | mg/mL |
| solution pH 3 | | | |
| 100 mM Citric | 2.5 mg | 2 mL | <1.3 mg/mL |
| acid buffer | | | |
| solution pH 4 | | | |
| 100 mM Citric | 2.6 mg | 2 mL | <1.3 mg/mL |
| acid buffer | | | |
| solution pH 5 | | | |
| 100 mM Citric | 2.4 mg | 2 mL | <1.2 mg/mL |
| acid buffer | | | |
| solution pH 7 | | | |
| 100 mM Citric | 2.4 mg | 2 mL | <1.2 mg/mL |
| acid buffer | | | |
| solution pH 9 | | | |

### Comparative Example 2

### Solubilizing Effect of Various Sugars on Compound A1 Hydrochloride

Each of the solutions (0.1 mL) shown in Table 2 was added to compound A1 hydrochloride (about 2 mg), and solubility was confirmed with eye observation. When insoluble materials were recognized, the solution (0.1 mL) was further added, and solubility was confirmed by eye observation in the same manner. When insoluble materials were still recognized, the solution was thereafter added in the amounts of 0.05 mL, 0.15 mL, 0.4 mL, 0.4 mL, 0.4 mL and 0.4 mL, respectively in this order, and solubility was confirmed by eye observation. The concentration of the compound A1 hydrochloride determined from the total amount of the solution added up to the time point at which insoluble materials were not recognized, was taken as the solubility.

A solubilizing effect exhibiting a solubility of 8 mg/mL or more was not obtained with the solutions of sugars in Table 2. In addition, the symbol “<” in the table indicates that insoluble materials were recognized at the time point where the solution was added up to a total amount of 2 mL.

TABLE 2

| | Weighed value | | |
|---|---|---|---|
| | of compound | Total amount | |
| | A1 | of added | |
| Solution | hydrochloride | solution | Solubility |
| Water for | 2.4 mg | 2 mL | <1.2 mg/mL |
| injection | | | |
| 5 w/v % D- | 2.2 mg | 2 mL | About 1.1 |
| mannitol in | | | mg/mL |
| water for | | | |
| injection | | | |
| 5 w/v % D- | 2.6 mg | 2 mL | About 1.3 |
| sorbitol in | | | mg/mL |
| water for | | | |
| injection | | | |
| 5 w/v % | 2.5 mg | 2 mL | About 1.3 |
| glucose in | | | mg/mL |
| water for | | | |
| injection | | | |
| 10 w/v % | 2.4 mg | 2 mL | About 1.2 |
| sucrose in | | | mg/mL |
| water for | | | |
| injection | | | |
| 10 w/v % | 2.7 mg | 2 mL | About 1.4 |
| maltose in | | | mg/mL |
| water for | | | |
| injection | | | |
| 10 w/v % | 2.5 mg | 2 mL | <1.3 mg/mL |
| Dextran 40 in | | | |
| water for | | | |
| injection | | | |

### Comparative Example 3

### Solubilizing Effect of Various Solubilizers on Compound A1 Hydrochloride

Each of the solutions (0.1 mL) shown in Table 3 was added to compound A1 hydrochloride (about 2 mg), and solubility was confirmed with eye observation. When insoluble materials were recognized, the solution (0.1 mL) was further added, and solubility was confirmed by eye observation in the same manner. When insoluble materials were still recognized, the solution was thereafter added in the amounts of 0.05 mL, 0.15 mL, 0.4 mL, 0.4 mL, 0.4 mL and 0.4 mL, respectively in this order, and solubility was confirmed by eye observation. The concentration of the compound A1 hydrochloride determined from the total amount of the solution added up to the time point at which insoluble materials were not recognized, was taken as the solubility.

A solubilizing effect exhibiting a solubility of 8 mg/mL or more was not obtained with the solutions of solubilizers in Table 3. In addition, the symbol “<” in the table indicates that insoluble materials were recognized at the time point where the solution was added up to a total amount of 2 mL.

TABLE 3

| | Weighed value | | | |
|---|---|---|---|---|
| | of compound | Total amount | | |
| | A1 | of added | | |
| Solution | hydrochloride | solution | | Solubility |
| Water for | 2.4 mg | 2 | mL | <1.2 mg/mL |
| injection | | | | |
| 100 w/v % | 2.4 mg | 2 | mL | <1.2 mg/mL |
| Ethanol | | | | |
| 100 w/v % | 2.1 mg | 2 | mL | <1.1 mg/mL |
| Polyethylene | | | | |
| glycol 300 | | | | |
| 100 w/v % | 2.5 mg | 2 | mL | <1.3 mg/mL |
| Polyethylene | | | | |
| glycol 400 | | | | |
| 10 w/v % | 2.6 mg | 1.2 | mL | About 2.2 |
| Polysorbate | | | | mg/mL |
| 80 in water | | | | |
| for injection | | | | |
| 10 w/v % | 2.2 mg | 1.2 | mL | About 1.8 |
| Polysorbate | | | | mg/mL |
| 20 in water | | | | |
| for injection | | | | |
| 100 w/v % | 2.3 mg | 2 | mL | <1.2 mg/mL |

Soybean oil

### Example 1

0.25 mL of a 30 w/v % solution of sulfobutyl ether-β-cyclodextrin sodium salt (Captisol; registered trademark, clinical grade) in water for injection was added to 2.3 mg of compound A1 hydrochloride to prepare a solution. Upon eye observation, the solution was clear, and no insoluble materials were recognized.

### Example 2

0.25 mL of a 30 w/v % solution of 2-hydroxypropyl-β-cyclodextrin in water for injection was added to 2.6 mg of compound A1 hydrochloride to prepare a solution. Upon eye observation, the solution was clear, and no insoluble materials were recognized.

### Example 3

0.25 mL of a 30 w/v % solution of maltosyl-β-cyclodextrin in water for injection was added to 2.7 mg of compound A1 hydrochloride to prepare a solution. Upon eye observation, the solution was clear, and no insoluble materials were recognized.

### Example 4

Sulfobutyl ether-β-cyclodextrin sodium salt (Captisol; registered trademark, research grade) was added and dissolved in water for injection, subsequently citric acid monohydrate was added and dissolved therein, and in the resulting solution, compound A1 hydrochloride was added and dissolved, so that the concentrations of the components in the solution would be 20 mg/mL for compound A1 (free form), 12 w/v % for sulfobutyl ether-β-cyclodextrin sodium salt (Captisol; registered trademark, research grade), and 40 mM for citric acid. Thereafter, the resulting solution was made up with water for injection so as to prepare a solution at about pH 3.

### Example 5

Sulfobutyl ether-β-cyclodextrin sodium salt (Captisol; registered trademark, research grade) was added and dissolved in water for injection, subsequently citric acid monohydrate was added and dissolved therein, and in the resulting solution, compound A1 hydrochloride was added and dissolved, so that the concentrations of the components in the solution would be 20 mg/mL for compound A1 (free form), 12 w/v % for sulfobutyl ether-β-cyclodextrin sodium salt (Captisol; registered trademark, research grade), and 40 mM for citric acid. Thereafter, the resulting solution was made up with water for injection so as to prepare a solution at about pH 3. Then, the solution was adjusted to about pH 2.5 with a small amount of hydrochloric acid, was prepared.

### Example 6

Sulfobutyl ether-β-cyclodextrin sodium salt (Captisol; registered trademark, research grade) was added and dissolved in water for injection, subsequently citric acid monohydrate was added and dissolved therein, and in the resulting solution, compound A1 hydrochloride was added and dissolved, so that the concentrations of the components in the solution would be 20 mg/mL for compound A1 (free form), 12 w/v % for sulfobutyl ether-β-cyclodextrin sodium salt (Captisol; registered trademark, research grade), and 40 mM for citric acid. Thereafter, the resulting solution was made up with water for injection so as to prepare a solution at about pH 3. Then, the solution was adjusted to about pH 3.5 with a small amount of sodium hydroxide, was prepared.

### Experimental Example 1

### Solubility Improving Effect of Various Cyclodextrin Derivatives on Compound A1 Hydrochloride

Each of the solutions (0.1 mL) shown in Table 4 was added to compound A1 hydrochloride (about 2 mg), and solubility was confirmed with eye observation. When insoluble materials were recognized, the solution (0.1 mL) was further added, and solubility was confirmed by eye observation in the same manner. When insoluble materials were still recognized, the solution was thereafter added in the amounts of 0.05 mL, 0.15 mL, 0.4 mL, 0.4 mL, 0.4 mL and 0.4 mL, respectively in this order, and solubility was confirmed by eye observation. The concentration of the compound A1 hydrochloride determined from the total amount of the solution added up to the time point at which insoluble materials were not recognized, was taken as the solubility.

Compared to the solubility in the water for injection, all of the various cyclodextrin derivative solutions shown in Table 4 exhibited remarkable solubility improving effect, a solubility of 8 mg/mL or more. In addition, the symbol “<” in the table indicates that insoluble materials were recognized at the time point where the solution was added up to a total amount of 2 mL.

TABLE 4

| | Weighed value | | |
|---|---|---|---|
| | of compound | Total amount | |
| | A1 | of added | |
| Solution | hydrochloride | solution | Solubility |
| Water for | 2.4 mg | 2 mL | <1.2 mg/mL |
| injection | | | |
| 30 w/v % | 2.3 mg | 0.25 mL | About 9.2 |
| Sulfobutyl | | | mg/mL |
| ether-β- | | | |
| cyclodextrin | | | |
| sodium salt | | | |
| (Captisol; | | | |
| registered | | | |
| trademark, | | | |
| clinical grade) | | | |
| in water for | | | |
| injection | | | |
| 30 w/v % 2- | 2.6 mg | 0.25 mL | About 10.4 |
| Hydroxypropyl- | | | mg/mL |
| β-cyclodextrin | | | |
| in water for | | | |
| injection | | | |
| 30 w/v % | 2.7 mg | 0.25 mL | About 10.8 |
| Maltosyl-β- | | | mg/mL |
| cyclodextrin in | | | |
| water for | | | |
| injection | | | |

### Experimental Example 2

### Solubility Improving Effect of Sulfobutyl ether-β-cyclodextrin Sodium Salt Solutions at Various pH Values on Compound A1 Hydrochloride

To each of the various solutions shown in Table 5, compound A1 hydrochloride was added in an amount exceeding the saturation concentration, and the mixture was shaken for 18 hours at 25° C. Subsequently, this solution was filtered in two stages through a hydrophilic filter having a pore size of 0.45 μm and a hydrophilic filter having a pore size of 0.22 μm, the filtrate was recovered, and the concentration of the compound A1 (free form) in the filtrate was measured by HPLC to examine solubility. It was found from the data of Table 5 that the addition of sulfobutyl ether-β-cyclodextrin sodium salt (Captisol; registered trademark, research grade) resulted in a significant increase in the solubility of compound A1.

TABLE 5

| Solution | Solubility |
|---|---|
| 40 mM Citric acid, water for injection, HCl, pH 2.5 | 8.0 mg/mL |
| 5 w/v % Sulfobutyl ether-β-cyclodextrin sodium | 29.2 mg/mL |
| salt (Captisol; registered trademark, research | |
| grade), 40 mM Citric acid, water for | |
| injection, HCl, pH 2.5 | |
| 40 mM Citric acid, water for injection, NaOH, | 0.3 mg/mL |
| pH 4 | |
| 5 w/v % Sulfobutyl ether-β-cyclodextrin sodium | 24.5 mg/mL |
| salt (Captisol; registered trademark, research | |
| grade), 40 mM Citric acid, water for | |
| injection, NaOH, pH 4 | |
| 40 mM Citric acid, water for injection, NaOH, | 0.1 mg/mL |
| pH 5 | |
| 5 w/v % Sulfobutyl ether-β-cyclodextrin sodium | 22.9 mg/mL |
| salt (Captisol; registered trademark, research | |
| grade), 40 mM Citric acid, water for | |
| injection, NaOH, pH 5 | |

### Experimental Example 3

### Solubility Improving Effect of Sulfobutyl ether-β-cyclodextrin Sodium Salt Solutions Having Various Concentrations on Compound A1 Hydrochloride

To each of the various solutions shown in Table 6, compound A1 hydrochloride was added in an amount exceeding the saturation concentration, and the mixture was shaken for 18 hours at 5° C. Subsequently, this solution was filtered in two stages through a hydrophilic filter having a pore size of 0.45 μm and a hydrophilic filter having a pore size of 0.22 μm, the filtrate was recovered, and the concentration of the compound A1 (free form) in the filtrate was measured by HPLC to examine solubility. It was found from the data of Table 6 that the addition of sulfobutyl ether-β-cyclodextrin sodium salt (Captisol; registered trademark, research grade) resulted in a significant increase in the solubility of compound A1.

TABLE 6

| Solution | Solubility |
|---|---|
| 40 mM Citric acid, water for injection, NaOH, | 0.1 mg/mL |
| pH 4 | |
| 2.5 w/v % Sulfobutyl ether-β-cyclodextrin | 13.0 mg/mL |
| sodium salt (Captisol; registered trademark, | |
| research grade), 40 mM Citric acid, water for | |
| injection, NaOH, pH 4 | |
| 5 w/v % Sulfobutyl ether-β-cyclodextrin sodium | 20.9 mg/mL |
| salt (Captisol; registered trademark, research | |
| grade), 40 mM Citric acid, water for | |
| injection, NaOH, pH 4 | |
| 7.5 w/v % Sulfobutyl ether-β-cyclodextrin | 27.6 mg/mL |
| sodium salt (Captisol; registered trademark, | |
| research grade), 40 mM Citric acid, water for | |
| injection, NaOH, pH 4 | |
| 10 w/v % Sulfobutyl ether-β-cyclodextrin sodium | 32.8 mg/mL |
| salt (Captisol; registered trademark, research | |
| grade), 40 mM Citric acid, water for | |
| injection, NaOH, pH 4 | |
| 12 w/v % Sulfobutyl ether-β-cyclodextrin sodium | 35.4 mg/mL |
| salt (Captisol; registered trademark, research | |
| grade)., 40 mM Citric acid, water for | |
| injection, NaOH, pH 4 | |
| 20 w/v % Sulfobutyl ether-β-cyclodextrin sodium | 43.6 mg/mL |
| salt (Captisol; registered trademark, research | |
| grade), 40 mM Citric acid, water for | |
| injection, NaOH, pH 4 | |
| 30 w/v % Sulfobutyl ether-β-cyclodextrin sodium | 49.9 mg/mL |
| salt (Captisol; registered trademark, research | |
| grade), 40 mM Citric acid, water for | |
| injection, NaOH, pH 4 | |

### Experimental Example 4

### Stability Improving Effect of Sulfobutyl ether-β-cyclodextrin Sodium Salt Solution on Compound A1 Hydrochloride

Sulfobutyl ether-β-cyclodextrin sodium salt (Captisol; registered trademark, research grade) was added and dissolved in water for injection, subsequently citric acid monohydrate was added and dissolved therein, and in the resulting solution, compound A1 hydrochloride was added and dissolved, so that the concentrations of the components in the solution would be 20 mg/mL for compound A1 (free form), 12 w/v % for sulfobutyl ether-β-cyclodextrin sodium salt (Captisol; registered trademark, research grade), and 40 mM for citric acid. Thereafter, the resulting solution was made up with water for injection so as to prepare a solution at about pH 3. Then, the solution was adjusted to about pH 2.5 with a small amount of hydrochloric acid, and the solution was adjusted to about pH 3.5 with a small amount of sodium hydroxide, were prepared.

These solutions were respectively filtered through a hydrophilic filter having a pore size of 0.22 μm, and then were filled in colorless USP Type I glass vials in an amount of about 11 mL each. The vial head space was purged with nitrogen, and the vial was sealed with a rubber stopper and an aluminum overseal to produce solution injectable preparations at about pH 2.5, 3 or 3.5.

The produced injectable preparations were stored at 5° C., 25° C./60% RH, or 40° C./75% RH, and were tested for stability. The results in Table 7 were obtained. It was found from the data of Table 7 that the pharmaceutical composition of the present invention was very stable.

TABLE 7

| | | | | | | | Compound |
|---|---|---|---|---|---|---|---|
| | | | | Particulate | | | A1 (free |
| | | | | Matter*² | | | base) |
| | | | | (per | | | Concen- |
| Storage | | | | container) | | Assay | tration |
| Condition | | pH | FIM*¹ | ≧10 μm | ≧25 μm | (%) | (mg/mL) |
| Initial | | 2.5 | Passed | 3 | 0 | 100.1 | 20.0 |
| 5° C. | 3 M | 2.5 | Passed | 11 | 1 | 101.0 | 20.2 |
| 25° C./ | 3 M | 2.5 | Passed | 7 | 0 | 99.8 | 20.0 |
| 60% | | | | | | | |
| RH | | | | | | | |
| 40° C./ | 3 M | 2.5 | Passed | 7 | 0 | 100.0 | 20.0 |
| 75% | | | | | | | |
| RH | | | | | | | |
| Initial | | 3.0 | Passed | 16 | 1 | 99.7 | 19.9 |
| 5° C. | 3 M | 3.0 | Passed | 3 | 0 | 101.3 | 20.3 |
| 25° C./ | 3 M | 3.0 | Passed | 2 | 0 | 99.3 | 19.9 |
| 60% | | | | | | | |
| RH | | | | | | | |
| 40° C./ | 3 M | 3.0 | Passed | 9 | 1 | 100.5 | 20.1 |
| 75% | | | | | | | |
| RH | | | | | | | |
| Initial | | 3.5 | Passed | 1 | 0 | 100.0 | 20.0 |
| 5° C. | 3 M | 3.5 | Passed | 1 | 0 | 101.3 | 20.3 |
| 25° C./ | 3 M | 3.4 | Passed | 14 | 1 | 99.2 | 19.8 |
| 60% | | | | | | | |
| RH | | | | | | | |
| 40° C./ | 3 M | 3.4 | Passed | 1 | 0 | 99.0 | 19.8 |
| 75% | | | | | | | |
| RH | | | | | | | |

*¹Foreign insoluble matter: visually inspected under 3750 Lux illuminance
*²USP <788> Particulate Matter limit: not more than 6000 for ≧10 μm, not more than 600 for ≧25 μm

### Experimental Example 5

### Stability Improving Effect of Sulfobutyl ether-β-cyclodextrin Sodium Salt Solution on Compound A1 Hydrochloride

The preparation at pH 3 produced in Experimental Example 4 was diluted to 100-folds with physiological saline and a 5% glucose solution to obtain a solution having a concentration of compound A1 (free form) of 0.2 mg/mL. The solution was stored under shaded conditions at 25° C. for 24 hours, and stability was examined. The results in Table 8 were obtained. It was found from the data of Table 8 that the pharmaceutical composition of the present invention was very stable even after dilution with physiological saline and a 5% glucose solution.

TABLE 8

| | | | | | | Compound |
|---|---|---|---|---|---|---|
| | | | | Particulate | | A1 (free |
| | | | | Matter*² | | base) |
| | | | | (per | | Content |
| | Storage | | | container) | | (% of |
| | Condition | pH | FIM*¹ | ≧10 μm | ≧25 μm | initial) |
| 0.9% | Initial | 3.6 | Passed | 203 | 0 | 100 |
| NaCl | 25° C., 24 hrs | 3.6 | Passed | 417 | 17 | 99.4 |
| | Protect from | | | | | |
| | light | | | | | |
| 5% | Initial | 3.8 | Passed | 467 | 3 | 100 |
| Glucose | 25° C., 24 hrs | 3.8 | Passed | 567 | 20 | 101.0 |
| | Protect from | | | | | |
| | light | | | | | |

*¹Foreign insoluble matter: visually inspected under 3750 Lux illuminance
*²USP <788> Particulate Matter limit: not more than 6000 for ≧10 μm, not more than 600 for ≧25 μm

### INDUSTRIAL APPLICABILITY

The pharmaceutical composition of the present invention contains a water-insoluble or slightly water-soluble compound (I), a salt thereof or a prodrug thereof, having improved solubility, stability property and the like. The pharmaceutical composition of the present invention is useful as a Kinase inhibitor for the prophylaxis or treatment of diseases such as cancer, inflammation, inflammatory bowel disease, psoriasis, transplant rejection, amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss, contraceptive medication, mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment and cognitive impairment, androgenetic alopecia, dementia related diseases, Alzheimer's Disease, conditions associated with kinases, arthritis and the like.

This application is based on a U.S. provisional patent application No. 61/032,165, the contents of which are incorporated in full herein by this reference.

1. A pharmaceutical composition comprising;
i) a compound represented by formula (I): 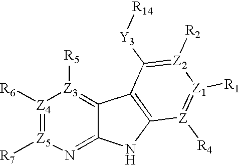
wherein
Z, Z₁, Z₂, Z₃, Z₄ and Z₅ are each independently selected from the group consisting of C and N;
R₁ is —Y₁—R₁₂, or R₁ is absent when Z₁ is N;
R₂ is —Y₂—R₁₃, or R₂ is absent when Z₂ is N, or R₁ and
R₂ are taken together to form a substituted or unsubstituted ring;
Y₁, Y₂ and Y₃ are each independently absent or a linker providing 1 or 2 atom separation between R₁₂, R₁₃ or R₁₄ and the ring to which Y₁, Y₂ or Y₃ is attached, wherein the atoms of the linker providing the separation are selected from the group consisting of carbon, oxygen, nitrogen, and sulfur;
R₄ is selected from the group consisting of hydrogen, halo, nitro, cyano, thio, oxy, hydroxy, carbonyloxy, alkoxy, carbonyl, amino, (C₁₋₅)alkylamino, (C₁₋₅)alkyl, halo(C₁₋₅)alkyl, carbonyl(C₁₋₃)alkyl, sulfonyl(C₁₋₃)alkyl, amino(C₁₋₅)alkyl, aryl(C₁₋₅)alkyl, heteroaryl(C₁₋₅)alkyl, (C₃₋₆)cycloalkyl and hetero(C₃₋₆)cycloalkyl, each substituted or unsubstituted, with the proviso that R₄ is absent when the atom to which it is bound is N;
R₅ and R₆ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, with the proviso that each of R₅ and R₆ is absent when the atom to which it is bound is N;
R₇ is selected from the group consisting of hydrogen, halo, hydroxy, alkoxy, amino and (C₁₋₅)alkyl, each substituted or unsubstituted, with the proviso that R₇ is absent when the atom to which it is bound is N;
R₁₂ and R₁₃ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₉₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₂ and R₁₃ are taken together to form a substituted or unsubstituted ring; and
R₁₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted,
with the provisos that (a) —Y₃—R₁₄ is not H when Z, Z₁, Z₂, Z₃ and Z₅ are all C; R₅ is a substituted amino group; and R₂ is methoxy or R₇ is methyl or amino; and (b) R₁₄ is not 3-chlorophenyl when R₁, R₅, R₆ and R₇ are each H; Z and Z₂ are each N; R₂ and R₄ are absent; Z₁, Z₃, Z₄ and Z₅ are all C; and Y₃ is NH,
a salt thereof or a prodrug thereof,
ii) at least one cyclodextrin derivative and
iii) water.

2. The composition of claim 1, wherein the compound represented by the formula (I) described in claim 1 is 5-(3-(ethylsulfonyl)phenyl)-3,8-dimethyl-N-(1-methylpiperidin-4-yl)-9H-pyrido[2,3-b]indole-7-carboxamide.

3. The composition of claim 1, which is used as an injectable composition.

4. The composition of claim 1, which is a non-emulsified composition.

5. The composition of claim 1, further comprising at least one pharmaceutically acceptable substance selected from a solubilizer, a suspending agent, an isotonic agent, a buffering agent, a local anesthetics and a pH adjusting agent.

6. The composition of claim 1, wherein pH of the composition is 2 to 5.

7. The composition of claim 1, which comprises about 0.01 to about 10 mol of the cyclodextrin derivative per 1 mol of the compound of the formula (I) described in claim 1.

8. The composition of claim 1, wherein the cyclodextrin derivative is at least one selected from a hydroxyalkyl cyclodextrin, a glucosyl cyclodextrin, a maltosyl cyclodextrin and a sulfoalkyl ether cyclodextrin.

9. The composition of claim 1, wherein the cyclodextrin derivative is at least one selected from a hydroxyalkyl cyclodextrin, a glucosyl cyclodextrin, a maltosyl cyclodextrin and a sulfoalkyl ether cyclodextrin, and a salt thereof.

10. The composition of claim 1, wherein the cyclodextrin derivative is sulfobutyl ether-J-cyclodextrin sodium salt.

11. The composition of claim 1, wherein the content of the cyclodextrin derivative in the pharmaceutical composition is about 0.01 to about 90 w/v %.

12. The composition of claim 1, which is an agent for preventing or treating of cancer, inflammation, inflammatory bowel disease, psoriasis, transplant rejection, amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss, contraceptive medication, mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment, cognitive impairment, androgenetic alopecia, dementia related diseases, Alzheimer's Disease or conditions associated with kinases.

13. A pharmaceutical composition comprising;
i) 5-(3-(ethylsulfonyl)phenyl)-3,8-dimethyl-N-(1-methylpiperidin-4-yl)-9H-pyrido[2,3-b]indole-7-carboxamide or a salt thereof,
ii) sulfobutyl ether-β-cyclodextrin or a salt thereof, and
iii) water.

14. A pharmaceutical composition comprising;
i) 5-(3-(ethylsulfonyl)phenyl)-3,8-dimethyl-N-(1-methylpiperidin-4-yl)-9H-pyrido[2,3-b]indole-7-carboxamide or a salt thereof,
ii) sulfobutyl ether-β-cyclodextrin or a salt thereof,
iii) water and
iv) citric acid,
which is an injectable composition, having a pH of about 2 to about 4.

15. The composition of claim 14, which is prepared by a method comprising;
i) dissolving a) 5-(3-(ethylsulfonyl)phenyl)-3,8-dimethyl-N-(1-methylpiperidin-4-yl)-9H-pyrido[2,3-b]indole-7-carboxamide or a salt thereof, b) sulfobutyl ether-β-cyclodextrin or a salt thereof and c) citric acid in water to obtain a solution, and
ii) adjusting pH of the solution to about 2 to about 4.

16. A pharmaceutical composition comprising;
i) 5 to 40 mg/mL of 5-(3-(ethylsulfonyl)phenyl)-3,8-dimethyl-N-(1-methylpiperidin-4-yl)-9H-pyrido[2,3-b]indole-7-carboxamide or a salt thereof,
ii) 2 to 30 w/v % of sulfobutyl ether-β-cyclodextrin or a salt thereof,
iii) water and
iv) 10 to 100 mmol/L of citric acid,
which is an injectable composition, having a pH of about 2 to about 4.

17. The composition of any one of claims 13-16, wherein the sulfobutyl ether-β-cyclodextrin or a salt thereof is sulfobutyl ether-β-cyclodextrin sodium salt.

18. A pharmaceutical composition comprising;
i) a compound represented by formula (I): 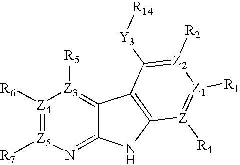
wherein
Z, Z₁, Z₂, Z₃, Z₄ and Z₅ are each independently selected from the group consisting of C and N;
R₁ is —Y₁—R₁₂, or R₁ is absent when Z₁ is N;
R₂ is —Y₂—R₁₃, or R₂ is absent when Z₂ is N, or R₁ and
R₂ are taken together to form a substituted or unsubstituted ring;
Y₂ and Y₃ are each independently absent or a linker providing 1 or 2 atom separation between R₁₂, R₁₃ or
R₁₄ and the ring to which Y₁, Y₂ or Y₃ is attached, wherein the atoms of the linker providing the separation are selected from the group consisting of carbon, oxygen, nitrogen and sulfur;
R₄ is selected from the group consisting of hydrogen, halo, nitro, cyano, thio, oxy, hydroxy, carbonyloxy, alkoxy, carbonyl, amino, (C₁₋₅)alkylamino, (C₁₋₅)alkyl, halo(C₁₋₆)alkyl, carbonyl(C₁₋₃)alkyl, sulfonyl(C₁₋₃)alkyl, amino(C₁₋₆)alkyl, aryl(C₁₋₅)alkyl, heteroaryl(C₁₋₆)alkyl, (C₃₋₆)cycloalkyl and hetero(C₃₋₆)cycloalkyl, each substituted or unsubstituted, with the proviso that R₄ is absent when the atom to which it is bound is N;
R₅ and R₆ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃) alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₃₋₁₂)bicycloaryl and hetero(C₄₋₁₂)bicycloaryl, each substituted or unsubstituted, with the proviso that each of R₅ and R₆ is absent when the atom to which it is bound is N;
R₇ is selected from the group consisting of hydrogen, halo, hydroxy, alkoxy, amino and (C₁₋₅)alkyl, each substituted or unsubstituted, with the proviso that R₇ is absent when the atom to which it is bound is N;
R₁₂ and R₁₃ are each independently selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₉₋₁₂)bicycloaryl(C₁₋₅)alkyl, hetero(C₈₋₁₂)bicycloaryl(C₁₋₅) alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₉₋₁₂)bicycloaryl and hetero(C₄₋₁₂)bicycloaryl, each substituted or unsubstituted, or R₁₂ and R₁₃ are taken together to form a substituted or unsubstituted ring; and
R₁₄ is selected from the group consisting of hydrogen, halo, nitro, cyano, thio, oxy, hydroxy, alkoxy, aryloxy, heteroaryloxy, carbonyl, amino, (C₁₋₁₀)alkylamino, sulfonamido, imino, sulfonyl, sulfinyl, (C₁₋₁₀)alkyl, halo(C₁₋₁₀)alkyl, carbonyl(C₁₋₃)alkyl, thiocarbonyl(C₁₋₃)alkyl, sulfonyl(C₁₋₃)alkyl, sulfinyl(C₁₋₃)alkyl, amino(C₁₋₁₀)alkyl, imino(C₁₋₃)alkyl, (C₃₋₁₂)cycloalkyl(C₁₋₅)alkyl, hetero(C₃₋₁₂)cycloalkyl(C₁₋₅)alkyl, aryl(C₁₋₁₀)alkyl, heteroaryl(C₁₋₅)alkyl, (C₃₋₁₂)bicycloaryl(C₁₋₅)alkyl, hetero(C₈₋₁₂)bicycloaryl(C₁₋₅)alkyl, (C₃₋₁₂)cycloalkyl, hetero(C₃₋₁₂)cycloalkyl, (C₉₋₁₂)bicycloalkyl, hetero(C₃₋₁₂)bicycloalkyl, aryl, heteroaryl, (C₃₋₁₂)bicycloaryl and hetero(C₄₋₁₂)bicycloaryl, each substituted or unsubstituted,
with the provisos that (a) —Y₃—R₁₄ is not H when Z, Z₁, Z₂, Z₃ and Z₅ are all C; R₅ is a substituted amino group; and R₂ is methoxy or R₇ is methyl or amino; and (b) R₁₄ is not 3-chlorophenyl when R₁, R₅, R₆ and R₇ are each H; Z and Z₂ are each N; R₂ and R₄ are absent; Z₁, Z₃, Z₄ and Z₅ are all C; and Y₃ is NH,
a salt thereof or a prodrug thereof, and
ii) sulfobutyl ether-β-cyclodextrin sodium salt.

19. A pharmaceutical composition comprising;
i) 5-(3-(ethylsulfonyl)phenyl)-3,8-dimethyl-N-(1-methylpiperidin-4-yl)-9H-pyrido[2,3-b]indole-7-carboxamide or a salt thereof, and
ii) sulfobutyl ether-f-cyclodextrin or a salt thereof.

20. A pharmaceutical composition comprising;
i) 5-(3-(ethylsulfonyl)phenyl)-3,8-dimethyl-N-(1-methylpiperidin-4-yl)-9H-pyrido[2,3-b]indole-7-carboxamide or a salt thereof, and
ii) sulfobutyl ether-β-cyclodextrin sodium salt.

21. A method for preventing or treating cancer, inflammation, inflammatory bowel disease, psoriasis, transplant rejection, amyotrophic lateral sclerosis, corticobasal degeneration, Down syndrome, Huntington's Disease, Parkinson's Disease, postencephelatic parkinsonism, progressive supranuclear palsy, Pick's Disease, Niemann-Pick's Disease, stroke, head trauma and other chronic neurodegenerative diseases, Bipolar Disease, affective disorders, depression, schizophrenia, cognitive disorders, hair loss, contraceptive medication, mild Cognitive Impairment, Age-Associated Memory Impairment, Age-Related Cognitive Decline, Cognitive Impairment No Dementia, mild cognitive decline, mild neurocognitive decline, Late-Life Forgetfulness, memory impairment, cognitive impairment, androgenetic alopecia, dementia related diseases, Alzheimer's Disease or conditions associated with kinases, which comprises administrating an effective amount of the composition of claim 1 to a mammalian species in need thereof.

22. A method of improving solubility in water of the compound of the formula (I) described in claim 1, a salt thereof or a prodrug thereof, which comprises combining the compound of the formula (I), a salt thereof or a prodrug thereof with at least one cyclodextrin derivative.

23. A method of improving stability in water of the compound of the formula (I) described in claim 1, a salt thereof or a prodrug thereof, which comprises combining the compound of the formula (I), a salt thereof or a prodrug thereof with at least one cyclodextrin derivative.
